(19) United States
(12) Patent Application Publication
(10) Pub. No.: US 2008/0025983 A1
(43) Pub. Date: Jan. 31, 2008

(54) Methods of screening based on the EGF receptor crystal structure

(75) Inventors: Timothy Edward Adams, Lower Plenty (AU); Antony Wilks Burgess, Camberwell (AU); Thomas Charles Elleman, Westmeadows (AU); Thomas Peter John Garrett, Brunswick (AU); Robert Nicholas Jorissen, Keysborough (AU); Meizhen Lou, Scoresby (AU); George Oscar Lovrecz, North Balwyn (AU); Neil Moreton McKern, Lilydale (AU); Edouard Collins Nice, St. Kilda (AU); Colin Wesley Ward, Carlton North (AU)

Correspondence Address: FOLEY AND LARDNER LLP;SUITE 500, 3000 K STREET NW, WASHINGTON, DC, 20007, US

(73) Assignees: Commonwealth Scientific and Industrial Research Organisation; Walter & Eliza Hall Institute of Medical Research; Ludwig Institute for Cancer Research

(21) Appl. No.: 11/882,679

(22) Filed: Aug. 3, 2007

Related U.S. Application Data
(62) Division of application No. 10/485,683, filed on Jul. 14, 2004, now abandoned.
(60) Provisional application No. 60/335,393, filed on Nov. 1, 2001.
(60) Provisional application No. 60/336,560, filed on Nov. 1, 2001.
(60) Provisional application No. 60/388,171, filed on Jun. 11, 2002.

(30) Foreign Application Priority Data
Aug. 3, 2001 (AU) PR 6827
Aug. 3, 2001 (AU) PR 6828
May 31, 2002 (AU) PS 2731

(51) Int. Cl. C07K 16/22; A61K 39/395

(52) U.S. Cl. 424/139.1; 530/387.9

(57) ABSTRACT

This invention relates to the structure of members of the epidermal growth factor (EGF) receptor family and to receptor/ligand interactions. In particular, it relates to the field of using the EGF receptor family structure to select and screen for compounds that inhibit the formation of active receptor dimers.

### FIELD OF THE INVENTION

This invention relates to the structure of members of the epidermal growth factor (EGF) receptor family and to receptor/ligand interactions. In particular, it relates to the field of using the EGF receptor family structure to select and screen for compounds that inhibit the formation of active receptor dimers.

### BACKGROUND OF THE INVENTION

Epidermal growth factor is a small polypeptide growth factor that stimulates marked proliferation of epithelial tissues and is a member of a larger family of structurally related growth factors such as transforming growth factor α (TGFα), amphiregulin, betacellulin, heparin-binding EGF and some viral gene products. Abnormal EGF family signalling is a characteristic of certain cancers (Yarden and Sliwkowski, 2001, Nature Reviews Mol Cell Biol. 2, 127-37; Soler and Carpenter, 1994 In Nicola, N. (ed) “Guidebook to Cytokines and their Receptors”, Oxford Univ. Press, Oxford, pp 194-197; Walker and Burgess, 1994, In Nicola, N. (ed) “Guidebook to Cytokines and their Receptors”, Oxford Univ. Press, Oxford, pp 198-201).

The epidermal growth factor receptor (EGFR) is the cell membrane receptor for EGF (Ullrich and Schlessinger, 1990, Cell 61, 203-212). The EGFR also binds other ligands that contain amino acid sequences classified as the EGF-like motif. Other known ligands of the EGFR are amphiregulin (Shoyab et al., 1988, Proc Natl Acad Sci USA. 85: 6528-6532; Shoyab et al., 1989, Science. 243: 1074-1076.), heparin-binding epidermal growth factor receptor (Higashiyama et al., 1991, Science. 251: 936-939.), betacellulin (Sasada et al., 1993, Biochem Biophys Res Commun. 190: 1173-1179; Shing et al., 1993, Science. 259: 1604-1607.), epiregulin (Toyoda et al., 1995, J Biol. Chem. 270: 7495-7500; Toyoda et al., 1997, Biochem J. 326: 69-75.) and epigen (Strachan et al., 2001, J Biol Chem. 276: 18265-18271.). Among these ligands, the three-dimensional structures of EGF and TGFα have been determined by NMR (Montelione et al., 1986 PNAS 83(22): 8594-8; Campbell et al., 1989, Prog. Growth Factor Res. 1, 13-22). Upon binding of the ligand to the extracellular domain, the EGFR undergoes dimerization, which eventually leads to the activation of its cytoplasmic protein tyrosine kinase (Ullrich and Schlessinger, 1990, Cell 61, 203-212). The EGFR is also known as the ErbB-1 receptor and belongs to the type I family of receptor tyrosine kinases (Ullrich, and Schlessinger, 1990, Cell 61, 203-212). This group also includes the ErbB-2, ErbB-3 and ErbB-4 receptors. No high affinity ligand has yet been found for ErbB-2 (Olayioye et al., 2000, EMBO J. 19: 3159-3167.). The neuregulins are alternatively spliced proteins from one of at least four genes which contain an EGF-motif and bind to ErbB-3 and/or ErbB-4 (Olayioye et al., 2000, EMBO J. 19: 3159-3167). One of the neuregulins known as heregulin-1α or NDF was found to fold into an EGF-like fold by NMR (Nagata et al., 1994, EMBO J. 13, 3517-3523 and Jacobson et al., 1996, Biochemistry 36, 3402-3417). The EGFR ligands epiregulin, betacellulin and heparin-binding epidermal growth factor receptor also bind to ErbB-4 (Olayioye et al., 2000, EMBO J. 19: 3159-3167.)

The type II family of receptor tyrosine kinases consists of the insulin receptor (INSR), the insulin-like growth factor I receptor (IGF-1), and the insulin receptor-related receptor (Ullrich and Schlessinger, 1990, Cell 61, 203-212). Although the type II receptors consist of four chains (α₂β₂), both the extracellular portions of the receptors from the two families, as well as the tyrosine kinase portions, share significant sequence homology, suggesting a common evolutionary origin (Ullrich and Schlessinger, 1990, Cell 61, 203-212, and Bajaj et al., 1987, Biochim. Biophys. Acta 916, 220-226).

The 621 amino acid residues of the extracellular domain of the human EGFR (sEGFR) can be subdivided into four domains as follows: L1, S1, L2 and S2, where L and S stand for “large” and “small” domains, respectively (Bajaj et al., 1987, Biochim. Biophys. Acta 916, 220-226, see FIG. 2). The L1 and L2 domains are homologous, as are the S1 and S2 domains.

Ligand-induced dimerization was first reported for the EGF receptor (Schlessinger, 1980, Trends Biochem Sci 13, 443-447) and now is widely accepted as a general mechanism for the transmission of growth stimulatory signals across the cell membrane. Although many biochemical experiments have been performed to reveal the molecular mechanism of receptor dimerization (Lemmon et al., 1997, EMBO J. 16, 281-294 and Tzabar et al., 1997, EMBO J. 16, 4938-4950 and Lax et al., 1991, J. Biol. Chem. 266, 13828-13833), the molecular mechanism by which monomeric ligands induce dimerization is still unknown for members of the EGFR family. Single particle averaging of electron microscopic images suggests that the overall shape of the sEGFR is four-lobed and doughnut-like (Lax et al., 1991, J. Biol. Chem. 266, 13828-13833). Small angle x-ray scattering also indicates that the sEGFR can be approximated by a flattened sphere with long diameters of 110 Å and a short diameter of 20 Å (Lemmon et al., 1997, EMBO J. 16, 281-294). The crystallization of sEGFR in complex with EGF has been published (Günther et al., 1990, J. Biol. Chem. 265, 22082-22085; Degenhardt et al., 1998, Acta Crystallogr. D Biol. Crystallogr. 54:999-1001), but the structure has not yet been reported, despite a decade of effort by many groups.

One EGF receptor ligand, TGF-α has been observed to be overproduced in keratinocyte cells which are subject to psoriasis (Turbitt et al., 1990, J. Invest. Dermatol. 95(2), 229-232; Higashimyama et al., 1991, J. Dermatol., 18(2), 117-119; Elder et al, 1990, 94(1), 19-25). The overproduction of at least one other EGF receptor ligand, amphiregulin, has also been implicated in psoriasis. (Piepkorn, 1996, Am. J. Dermatopath., 18(2), 165-171). Molecules that inhibit the EGF receptor have been shown to inhibit the proliferation of both normal keratinocytes (Dvir et al., 1991, J. Cell Biol., 113(4), 857-865) and psoriatic keratinocytes. (Ben-Bassat et al., 1995, Exp. Dermatol., 4(2), 82-88). These findings indicate that EGF receptor antagonists may be useful in the treatment of psoriasis.

Many cancer cells express constitutively active EGFR (Sandgreen et al., 1990, Cell, 61:1121-135; Karnes et al., 1992, Gastroenterology, 102:474-485) or other EGFR family members (Hynes, 1993, Semin. Cancer Biol. 4:19-26). Elevated levels of activated EGFR occur in bladder, breast, lung and brain tumours (Harris, et al., 1989, In Furth & Greaves (eds) The Molecular Diagnostics of human cancer. Cold Spring Harbor Lab. Press, CSH, NY, pp 353-357). Antibodies to EGFR can inhibit ligand activation of EGFR (Sato et al., 1983 Mol. Biol. Med. 1:511-529) and the growth of many epithelial cell lines (Aboud-Pirak et al., 1988, J. Natl Cancer Inst. 85:1327-1331). Patients receiving repeated doses of a humanised chimeric anti-EGFR monoclonal antibody (Mab) showed signs of disease stabilization. The large doses required and the cost of production of humanised Mab is likely to limit the application of this type of therapy. These findings indicate that the development of EGF receptor antagonists will be attractive anticancer agents.

### SUMMARY OF THE INVENTION

The present inventors have now obtained three-dimensional structural information concerning a complex of human epidermal growth factor receptor (EGFR) residues 1-501 with human TGFα. In the complex each ligand only contacts one receptor and each receptor fragment contacts only one ligand. The receptor dimer seen in the crystals is a back-to-back dimer (S1 to S1). The co-ordinates for the EGF receptor in back-to-back dimer configuration are shown in Appendix I and Appendix II. Appendix II is a refined version of the co-ordinates presented in Appendix I.

The information presented in this application can be used to predict the structure of related members of the EGF receptor family and the nature of the dimers formed by these receptors. This information can be used to develop compounds which interact with members of the EGF receptor family for use in therapeutic applications.

Accordingly, in a first aspect the present invention provides a method of selecting or designing a compound that interacts with a receptor of the EGF receptor family and modulates an activity associated with the receptor, the method comprising

(a) assessing the stereochemical complementarity between the compound and a topographic region of the receptor, wherein the receptor comprises:

(i) amino acids 1-501 of the EGF receptor positioned at atomic coordinates as shown in Appendix I or Appendix II, or structural coordinates having a root mean square deviation from the backbone atoms of said amino acids of not more than 1.5 Å;

(ii) one or more subsets of said amino acids related to the coordinates shown in Appendix I or Appendix II by whole body translations and/or rotations; or

(iii) amino acids present in the amino acid sequence of a receptor of the EGF receptor family, which form an equivalent three-dimensional structure to that of amino acids 1-501 of the EGF receptor positioned at atomic coordinates substantially as shown in Appendix I or Appendix II, or structural coordinates having a root mean square deviation from the backbone atoms of said amino acids of not more than 1.5 Å, or one or more subsets thereof,

(b) obtaining a compound which possesses stereochemical complementarity to a topographic region of the receptor; and

(c) testing the compound for its ability to modulate an activity associated with the receptor.

In a preferred embodiment of the first aspect, the structural coordinates have a root mean square deviation from the backbone atoms of said amino acids of not more than 1.0 Å and more preferably not more than 0.7 Å.

In one embodiment of the first aspect, the subset of amino acids is selected from the group consisting of the subset of amino acids representing the L1 domain, the subset of amino acids representing the L2 domain and the subset of amino acids representing the S1 domain.

In another embodiment, the subset of amino acids relates to a semi-rigid domain within the EGF receptor, such as a domain based on or about residues 1-84; 191-237; 238-271; 271-284; 285-305 or 313-501; or an equivalent domain of another member of the EGF receptor family.

By “stereochemical complementarity” we mean that the compound or a portion thereof makes a sufficient number of energetically favourable contacts with the receptor as to have a net reduction of free energy on binding to the receptor.

From the information provided in Appendix I and Appendix II it can be seen that TGFα interacts with residues 1-501 of EGFR such that residues 3-5, 22, 24, 26, 27, 29-34, 36, 38-41, 43, 44, 47 and 49 of TGFα interact with residues 11-18, 20, 22, 26, 29, 30, 45, 69, 89, 90, 98, 99, 101-103, 125, 127 and 128 of L1 of EGFR and residues 8, 9, 11-15, 17, 18, 38, 39, 42 and 44-50 of TGFα interact with residues 325, 346, 348-350, 353-358, 382, 384, 408, 409, 411, 412, 415, 417, 418, 438, 440, 465 and 467 of L2 of EGFR.

Two residues or groups of residues are taken to “interact” when the solvent accessible surface calculated for one set of residues is reduced if it is recalculated in the presence of the other set of residues. The solvent accessible surface is defined by Lee. B and Richards, F. M. (1971) J. Mol. Biol. 55:379-400 using a probe radius of 1.4 Å.

The ligand binding surfaces of EGFR are therefore defined by residues 11-18, 20, 22, 26, 29, 30, 45, 69, 89, 90, 98, 99, 101-103, 125, 127 and 128 of L1 and residues 325, 346, 348-350, 353-358, 382, 384, 408, 409, 411, 412, 415, 417, 418, 438, 440, 465 and 467 of L2. It is believed that corresponding regions of other members of the EGF receptor family will also be involved in the binding of their natural ligand.

Accordingly, in one embodiment of the first aspect the compound is selected or designed to interact with a member of the EGF receptor family in a manner such as to interfere with the binding of natural ligand to:—

(i) one or more of the residues of EGFR selected from the group consisting of 11-18, 20, 22, 26, 29, 30, 45, 69, 89, 90, 98, 99, 101-103, 125, 127, 128, 325, 346, 348-350, 353-358, 382, 384, 408, 409, 411, 412, 415, 417, 418, 438, 440, 465 and 467 and combinations thereof; or

(ii) the corresponding region of other members of the EGF receptor family.

The compound may interfere with ligand binding to one or more of the specified residues in a number of ways. For example the compound may bind or interact with the receptor at or near one or more of the specified residues or corresponding regions and by steric overlap and/or electrostatic repulsion prevent natural ligand binding. Alternatively the compound may bind to the receptor so as to interfere allosterically with natural ligand binding. For example the compound may bind to the L1 and L2 domains in manner such as to decrease the “gap” between the L1 and L2 domains thereby preventing access of the ligand to one or more of the specified residues.

Alternatively the compound may bind to the receptor so as to interfere allosterically with natural ligand binding. For example:—

(i) The compound may bind to the L1 and L2 domains in manner such as to decrease the “gap” between the L1 and L2 domains thereby preventing access of the ligand to one or more of the specified residues.

(ii) The compound may bind at or near the interface between S1 and either L1 or L2 domains to thereby perturb the domain associations as shown in Appendix I and II for the signalling competent ligand-receptor complex.

(iii) The compound may bind at a site remote from the ligand-binding site but disturb the receptor structure so as to reduce the affinity of ligand binding.

Sites for allosteric interference lie within 5 Å of atomic positions listed in Appendices III and IV.

It is presently preferred, however, that the compound binds or interacts with the receptor at or near one or more of the specified residues or within the corresponding region.

Accordingly in one embodiment of the first aspect, the receptor is EGFR and topographic region of EGFR to which the compound has stereochemical complementarity is the ligand binding surface defined by amino acids 11-18, 20, 22, 26, 29, 30, 45, 69, 89, 90, 98, 99, 101-103, 125, 127 and 128, and/or the ligand binding surface defined by amino acids 325, 346, 348-350, 353-358, 382, 384, 408, 409, 411, 412, 415, 417, 418, 438, 440, 465 and 467.

The phrase “EGF receptor family” includes, but is not limited to, the EGF receptor, ErbB2, ErbB3 and ErbB4. In general, EGF receptor family molecules show similar domain arrangements and share significant sequence identity, preferably at least 40% identity.

The known natural ligands for these receptors are as follows:

EGFR EGF, TGFα, amphiregulin, betacellulin, epiregulin and heparin-binding EGF;

ErbB3 neuregulins 1 and 2;

ErbB4 neuregulins 1-4, betacellulin, epiregulin and heparin-binding EGF;

ErbB2 ErbB2 alone has not been reported to bind any ligand with high affinity but is preferred heterodimerisation partner for the other three EGF receptor family members, enhancing their affinities for their respective ligands and amplifying their signals.

The domain structure of the extracellular regions of the EGFR, ErbB-2, ErbB-3 and ErbB-4 are the same. The percentage identities of the sequences corresponding to the first 501 residues of the EGFR are 42-47% except for that for ErbB-3 and ErbB-4 which is 60%. Previously, it has been possible to construct models of ErbB-2, ErbB-3 and ErbB-4 based on the structure of the first three domains of the insulin-like growth factor receptor (Garrett et al., (1998) Nature. 394: 395-399.) as has been performed for the EGFR (Jorissen et al., (2000) Protein Sci. 9: 310-324.) where the sequence identity is approximately 25%. At the higher sequence identity between EGFR and the other EGFR family members, models can be constructed which are expected to have a smaller degree of error (Tramontano A. (1998) Methods. 14: 293-300).

A sequence alignment between the four EGFR family members is shown in FIG. 1. Using the information provided in Appendix I Appendix II and the sequence alignment models of other members of the EGF receptor family can be obtained using the methods described in the reference referred to above.

The structure of the TGFα-EGFR complex also allows construction of the binding of EGFR family ligands to be modelled. Several interactions between TGFα and the sEGFR501 suggest that the observed mode of binding is the same for the EGFR family members and their ligands. There are two mainchain-to-mainchain hydrogen bonds between the EGFR L1 domain and TGFα:EGFR Gln 16.N-TGFα Cys 32.O and Gln 16.O-TGFα Cys 34.N. The sidechain of conserved TGFα residue Arg 42 forms a salt bridge with the sidechain of conserved EGFR residue Asp 355.

The sequence alignment of ligands for EGF receptor family is set out in FIG. 2.

The approximate ligand binding regions of ErbB-2, ErbB-3 and ErbB-4 can be deduced using the alignment of their sequences to that of the EGFR (FIG. 1) and the EGFR sequences listed earlier (residues 11-18, 20, 22, 26, 29, 30, 45, 69, 89, 90, 98, 99, 101-103, 125, 127, 128, 325, 346, 348-350, 353-358, 382, 384, 408, 409, 411, 412, 415, 417, 418, 438, 440, 465 and 467). For ErbB-2 (whose N-terminal sequence is taken to be STQV), these residues are 9-16, 18, 20, 24, 27, 28, 43, 67, 87, 88, 96, 97, 99-101, 133, 135, 136, 333, 354, 359-358, 361-366, 390, 392, 416, 417, 419, 420, 423, 425, 426, 446, 448, 473 and 475. For ErbB-3 (whose N-terminal sequence is taken to be SEVG), these residues are 14-21, 23, 25, 29, 32, 33, 48, 72, 92, 93, 101, 102, 104-106, 129, 131, 132, 322, 343, 345-347, 350-355, 379, 381, 405, 406, 408, 409, 412, 414, 415, 436, 438, 464 and 466. For ErbB-4 (whose N-terminal sequence is taken to be QPSD), these residues are 13-20, 22, 24, 28, 31, 32, 47, 71, 91, 92, 100, 101, 103-105, 128, 130, 131, 326, 347, 349-351, 354-359, 383, 385, 409, 410, 411, 412, 415, 417, 418, 439, 441, 466 and 468. (Note that the N-termini correspond to the putative start of the mature proteins according to their entries in the SWISSPROT database at the time of writing.) There are expected to be minor differences in the amino acids of the EGFR family member (including EGFR) which make up the ligand binding site depending on the identity of the ligand and receptor. For example, the EGFR residue Gly 442 is not listed as part of the binding site for bound TGFα but has been implicated in the binding of EGF (Elleman et al., (2001) Biochemistry. 40: 8930-8939.). A comparative model of the EGF-EGFR 1-501 complex shows that part of the sidechain of EGF residue Arg 45 is close to EGFR Gly 442. (The small size of the TGFα Ala 46 sidechain prevents this contact in the TGFα-bound complex.) Other variations in the definition of the ligand binding site for the modelled EGFR family member-ligand complex may arise from the variation in the size of the so-called B-loop of some of the EGFR family ligands (Groenen et al., (1994) Growth Factors. 11: 235-257.).

In a preferred embodiment of the first aspect of the present invention, the method comprises selecting or designing a compound which has portions that match residues positioned on the ligand binding surface of EGFR defined by amino acids 11-18, 20, 26, 29, 30, 45, 69, 89, 90, 98, 99, 101-103, 125, 127 and 128, and/or the ligand binding surface of EGFR defined by amino acids 325, 346, 348-350, 353-358, 382, 384, 408, 409, 411, 412, 415, 417, 418, 438 and 465, or the corresponding regions of other members of the EGF receptor family.

By “match” we mean that the identified portions interact with the surface residues, for example, via hydrogen bonding or by enthalpy-reducing Van der Waals and Coulomb interactions which promote desolvation of the biologically active compound with the receptor, in such a way that retention of the compound by the receptor is favoured energetically.

In a further preferred embodiment of the first aspect, the stereochemical complementarity between the compound and the receptor is such that the compound has a Kd for the receptor site of less than 10⁻⁶M, more preferably the Kd value is less than 10⁻⁸M and more preferably less than 10⁻⁹M.

In preferred embodiments of the first aspect of the present invention, the compound is selected or modified from a known compound identified from a data base.

A second aspect of the present invention provides a method of selecting or designing a compound that inhibits the formation of active dimers of receptors of the EGF receptor family, the method comprising:

(a) assessing the stereochemical complementarity between the compound and a topographic region of the receptor, wherein the receptor comprises:

(i) amino acids 1-501 of the EGF receptor positioned at atomic coordinates as shown in Appendix I or Appendix II, or structural coordinates having a root mean square deviation from the backbone atoms of said amino acids of not more than 1.5 Å;

(ii) one or more subsets of said amino acids related to the coordinates shown in Appendix I or Appendix II by whole body translations and/or rotations; or

(iii) amino acids present in the amino acid sequence of a receptor of the EGF receptor family, which form an equivalent three-dimensional structure to that of amino acids 1-501 of the EGF receptor positioned at atomic coordinates substantially as shown in Appendix I or Appendix II, or structural coordinates having a root mean square deviation from the backbone atoms of said amino acids of not more than 1.5 Å, or one or more subsets thereof,

(b) obtaining a compound which possesses stereochemical complementarity to a topographic region of the receptor; and

(c) testing the compound for its ability to inhibit the formation of active dimers of the receptors.

From the information provided in Appendix I and Appendix II it can also be seen that in the EGF dimer residues 38, 86, 194, 195, 204, 205, 230, 239, 242-246, 248-253, 262-265, 275, 278-280, 282-288 and 318 of the first receptor of the dimer interact with residues 86, 193, 194, 204, 205, 229, 230, 239, 242, 244-246, 248-253, 262-265, 275, 278-280 and 282-287 of the second receptor of the dimer. It is believed that corresponding regions of other members of the EGF receptor family will also be involved in the formation of active dimers.

Accordingly, in a further preferred form the compound is selected or designed to interact with a member of the EGF receptor family in a manner such as to interfere with the formation of active dimers by inhibiting interaction of;

(i) residues 38, 86, 194, 195, 204, 205, 230, 239, 242-246, 248-253, 262-265, 275, 278-280, 282-288 and 318 of EGFR or the corresponding region of a member of the EGF receptor family; with

(ii) residues 86, 193, 194, 204, 205, 229, 230, 239, 242, 244-246, 248-253, 262-265, 275, 278-280 and 282-287 of EGFR or the corresponding region of a member of the EGF receptor family.

The compound may interfere with dimerization in a number of ways. For example the compound may bind to the EGFR at or near one or more of the specified residues and by steric overlap an/or electrostatic repulsion prevent dimerization. Alternatively the compound may bind to EGFR so as to interfere allosterically with dimer formation.

Accordingly in one preferred embodiment of the second aspect, the receptor is EGFR and the topographic region of the EGFR to which the compound, or a portion thereof, has stereochemical complementarity is the dimer interface defined by amino acids 38, 86, 194, 195, 204, 205, 230, 239, 242-246, 248-253, 262-265, 275, 278-280, 282-288 and 318 and/or the dimer interface defined by amino acids 86, 193, 194, 204, 205, 229, 230, 239, 242, 244-246, 248-253, 262-265, 275, 278-280 and 282-287.

The regions of ErbB-2, ErbB-3 and ErbB-4 involved in dimerization can also be deduced using the alignment of their sequences to that of the EGFR (FIG. 1) and the EGFR sequences listed earlier (residues 38, 86, 193-195, 204, 205, 229, 230, 239, 242-246, 248-253, 262-265, 275, 278-280, 282-288, 318). For ErbB-2 (whose N-terminal sequence is taken to be STQV), these residues are 36, 84, 201-203, 211, 212, 236, 237, 246, 249-253, 255-260, 269-272, 282, 285-287, 289-295, 326. For ErbB-3 (whose N-terminal sequence is taken to be SEVG), these residues are 41, 89, 193-195, 204, 205, 229, 230, 239, 242-246, 248-253, 262-265, 275, 278-279, 281-287, 317. For ErbB-4 (whose N-terminal sequence is taken to be QPSD), these residues are 40, 88, 195-197, 206, 207, 231, 232, 241, 244-248, 250-255, 264-267, 277, 280-281, 283-289, 319. (Note that the N-termini correspond to the putative start of the mature proteins according to their entries in the SWISSPROT database at the time of writing.)

The mode of dimerization seen in the crystal structure is consistent with homodimers and heterodimers of all four EGFR family members. Several residues which appear to be important for maintaining the dimer interface in EGFR are conserved in the EGFR family. The conserved Asn 247 makes sidechain-to-mainchain hydrogen bonds which help to maintain the structure of the loop which interacts with the other EGFR molecule in the dimer. Residues Tyr 251 and Phe 263 are involved in packing interactions across the interface; these residues are either tyrosine or phenylalanine in ErbB-2, ErbB-3 and ErbB-4. The side chain of the conserved residue Tyr 246 makes hydrophobic packing and hydrogen bonding interactions with the other EGFR in the dimer.

As used herein the term “dimer” is intended to cover both homodimers and heterodimers.

By “active dimer” we mean a dimeric form which causes signalling.

In a further embodiment of the second aspect of the present invention, the method comprises selecting or designing a compound which has portions that match residues positioned on the dimer interface of EGFR defined by amino acids 38, 86, 194, 195, 204, 205, 230, 239, 242-246, 248-253, 262-265, 275, 278-280, 282-288 and 318 or the corresponding regions of other members of the EGF receptor family and/or the dimer interface defined by amino acids 86, 193, 194, 204, 205, 229, 230, 239, 242, 244-246, 248-253, 262-265, 275, 278-280 and 282-287 or the corresponding regions of other members of the EGF receptor family.

In a preferred embodiment the compound is designed or selected to comprise a first domain which interacts with the dimer interface of a first EGF receptor family member and a second domain which interacts with the dimer interface of a second EGF receptor family member. As will be recognised such a compound will cross-link receptor and prevent formation of active dimers.

In a further preferred embodiment of the second aspect of the present invention, the stereochemical complementarity is such that the compound has a K_{d} for the receptor site of less than 10⁻⁶M. More preferably, the K_{d} value is less than 10⁻⁸M and more preferably less than 10⁻⁹M.

In preferred embodiments of the second aspect of the present invention, the compound is selected or modified from a known compound identified from a data base.

The information provided in Appendix I and Appendix II also reveals the portions of TGFα which are involved in receptor binding. With this information TGFα variants may be designed in which specific residues are modified or altered such that the variant retains is able to bind to one ligand binding surface but not the other. It would be expected that such a variant would compete with the natural ligand for binding to the receptor but that binding of the variant to the receptor would not lead to signalling. Such a variant would therefore be an antagonist. In a similar manner variants which would act as agonists could be designed. In this case the modifications or alterations would be selected such as to increase the strength of interaction between the receptor and the variant so as to lead to increased signalling.

In a similar manner to that described for TGFα, variants of other ligands of the EGF receptor family may also be designed.

Accordingly in a third aspect the present invention consists in a TGFα variant in which the sequence of TGFα is modified such that the ability to interact with L1 of EGFR is retained or increased and the ability to interact with L2 of EGFR is removed or decreased, or vice versa.

In a fourth aspect the present invention consists in a TGFα variant in which the sequence of TGFα is modified such that the ability to interact with L1 of EGFR is retained or increased and the ability to interact with L2 of EGFR is retained or increased, with the proviso that the binding to at least one of L1 or L2 is increased.

In a preferred embodiment of these aspects of the present invention the TGFαvariant is modified at one more of the positions selected from the group consisting of 3-5, 8, 9, 11-15, 17, 18, 22, 24, 26, 27, 29-34, 36 and 38-50.

In a fifth aspect the present invention consists in an EGF variant in which the sequence of EGF is modified such that the ability to interact with L1 of EGFR is retained or increased and the ability to interact with L2 of EGFR is removed or decreased, or vice versa.

In a sixth aspect the present invention consists in an EGF variant in which the sequence of EGF is modified such that the ability to interact with L1 of EGFR is retained or increased and the ability to interact with L2 of EGFR is retained or increased, with the proviso that the binding to at least one of L1 or L2 is increased.

By “variant” we mean that the natural sequence of EGF or TGFα has been modified by one or more point mutations, insertions of amino acids, deletions of amino acids or replacement of amino acids, in particular using non-natural amino acids such as D-isomers of natural amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methylamino acids, Cα-methylamino acids, Nα-methylamino acids, β-naphthalimo amino acids and amino acid analogues in general.

The information provided in Appendix I and Appendix II also reveals the portions of EGFR which are involved in dimer formation and the portions EGFR involved in ligand binding. With this information EGFR variants or fragments may be designed in which specific residues are modified or altered such that the variant or fragment retains the ability to form dimers with the EGFR and or bind ligand. It would be expected that such variant or fragments would compete with the natural receptors for dimerization or ligand binding but that dimerization of the variant or fragment with the receptor would not lead to signalling.

Accordingly in a seventh aspect the present invention consists in a polypeptide, the polypeptide comprising amino acids which interact with amino acids 38, 86, 193-195, 204, 205, 229, 230, 239, 242-246, 248-253, 262-265, 275, 278-280, 282-288, 318 of EGFR or the corresponding region of a member of the EGF receptor family, or which are involved in binding of natural ligand of the EGF receptor family.

In a preferred embodiment the polypeptide is based on the native sequence of EGFR but includes modifications such that the interaction between the polypeptide and the native receptor is preferred over the interaction between native receptors.

In a further preferred embodiment the polypeptide is based on the native sequence of EGFR but includes modifications such that the interaction between the polypeptide and the natural ligand is preferred over the interaction between the natural ligand and native receptor.

As will be understood by those skilled in this field knowledge of the structure of a protein complex is of assistance in the development of mutants of one of the proteins with enhanced affinity for its protein partner. Structural information can be used to select residues on one or more of the protein interfaces in the complex for alteration by methods such as site-directed mutagenesis or phage display. For example, amino acid positions in growth hormone which were allowed to vary were chosen in part from the crystal structure of the complex of growth hormone bound to two molecules of the human growth hormone extracellular region (Lowman and Wells (1993) J Mol Biol. 234: 564-578.). Using a model of the granulocyte colony-stimulating factor (G-CSF) receptor ligand binding domain, residues of the receptor were chosen for mutagenesis by analogy with the structure of human growth hormone bound to its receptors (Layton et al., (1997) J Biol Chem. 272: 29735-29741.). Some of the mutant G-CSF receptors were found to bind G-CSF with slightly enhanced affinity (Layton et al., (1997) J Biol Chem. 272: 29735-29741.). The structure of the complex could also be used to design mutations which would potentially increase the binding affinity, for example by increasing the amount of hydrogen bonds and/or van der Waals interactions across the interface.

The modification of protein residues to enhance protein binding affinity is not restricted to those residues in the relevant protein-protein interfaces. Modification of residues outside of an interface may lead to alterations due to changes in the long-range electrostatic interactions between the two interacting proteins which changes the rate of association and subsequently the equilibrium binding constant (Selzer and Schreiber (1999) J Mol Biol. 287: 409-419; Selzer et al., (2000) Nat Struct Biol. 7: 537-541.). The contribution of mutations to the association rate can be calculated and has been used to increase the association rate (without greatly changing the dissociation rate) and the affinity of β-lactanase inhibitory protein to TEM1 β-lactamase by a factor of 250 (Seizer et al., (2000) Nat Struct Biol. 7: 537-541.).

There are two proposed modes of antagonist action of appropriate extracellular fragments of EGFR family members. The first is ligand binding. The sEGFR501 binds EGF and TGFα with approximately 10 times higher affinity than the full length extracellular portion of the EGFR (Elleman et al., (2001) Biochemistry. 40: 8930-8939.). The second mode is the association of these proteins with full-length receptors. Recombinant forms of the EGFR and ErbB-2 which contain only the extracellular domain and transmembrane domain are able to inhibit EGF-induced signalling when expressed on cells which also express the full length EGF receptor (Kashles et al., (1991) Mol Cell Biol. 11: 1454-1463; Spivak-Kroizman et al., (1992) J Biol Chem. 267: 8056-8063; Qian et al., (1999) J Biol Chem. 274: 574-583.), suggesting that the recombinant proteins act in a dominant negative manner which involves their extracellular regions.

The structure of the EGFR complex can be used to design mutations for extracellular fragments of EGFR family. Structural models of the other EGFR family members can be constructed as previously described. Mutations can be made either by expressing mutant versions of EGFR 1-501 or its homologues in which residues have been mutated individually or as groups, or by using the structure to locate amino acid positions which can be changed using methods such as phage display or DNA shuffling. These mutants can be tested or selected for enhanced affinity relative to the extracellular fragment based on the wild type EGFR family member's amino acid sequence. The preferred EGFR amino acids which are candidates for mutation are as follows:—

(i) 11-18, 20, 22, 26, 29, 30, 45, 69, 89, 90, 98, 99, 101-103, 125, 127, 128, 325, 346, 348-350, 353-358, 382, 384, 408, 409, 411, 412, 415, 417, 418, 438, 440, 465 and 467, or

(ii) 38, 86, 193-195, 204, 205, 229, 230, 239, 242-246, 248-253, 262-265, 275, 278-280, 282-288, 318.

The relevant residues for other members of the EGF receptor family can be determined from sequence alignments.

Additionally, the mutation of residues which are outside of the relevant binding interface may also alter the binding affinity by changes in the long range electrostatic interactions. These changes can affect the rate of association between two interacting proteins without greatly changing the rate of dissociation, and hence change the equilibrium binding constant (Selzer and Schreiber (1999) J Mol Biol. 287: 409-419; Seizer et al., (2000) Nat Struct Biol. 7: 537-541.). In one example of increasing the affinity of binding by mutating residues outside of the protein-protein interface, selected residues of the β-lactamase inhibitory protein that were outside of the interface were mutated so as to change their charge e.g. a basic residue mutated to a neutral residue and then the affinity and rate constants of the mutant binding to TEM1 β-lactamase was measured. In one mutant, the change of four amino acids led to an enhancement of binding by a factor of more 250-fold (Selzer et al., (2000) Nat Struct Biol. 7: 537-541.). In this example, the authors specified a formula which predicted the changes in the association constant upon mutation to within a factor of two (Selzer et al., (2000) Nat Struct Biol. 7: 537-541.). In this way, the structure of the EGFR or a model of one other EGFR family members could be used to predict mutations that would likely lead to an enhancement of the rate of association of the relevant EGFR family extracellular fragment to its interacting protein. Calculation and subsequent visualization of the electrostatic isopotentials (e.g. Smith and Treutlein (1998) Protein Sci. 7: 886-896.) may assist the selection of residues to mutate in order to increase the protein's rate of association. The most likely candidate residues for mutation are those on the periphery of the interface and those outside of the interface but which are within a specified distance of the interacting protein and are not completely buried in the L1 or L2 domain (as judged by visual examination). Cysteine residues, which are needed for the maintenance of the EGFR structure were also excluded from the list. For the EGFR, the preferred residues are:

(i) 5, 6, 8-10, 19, 21-25, 28, 32, 33, 38, 39, 40, 42, 44, 47, 48, 50, 63, 64, 66, 68, 71, 73, 87, 88, 91-94, 96, 104-107, 109, 123, 130, 131, 151-160, 315-324, 326, 328, 329, 331, 332, 343, 344, 351, 359-363, 379, 380, 385, 387, 388, 394, 404-407, 410, 413, 420, 434-436, 440, 441, 443, 448, 449, 461-464, 466-468; or

(ii) 1-6, 8, 9, 11, 30, 35, 36, 39, 40, 60, 62-64, 82, 84, 85, 87-89, 94, 118, 120-122, 148, 187-193, 196-198, 200-203, 209-211, 213, 215, 217-221, 231-233, 235, 237, 238, 241, 243, 244, 247, 254-261, 266, 268-270, 272-274, 276, 277, 281, 289-297, 299-301, 303, 304, 311, 312, 314-317, 319-323, 335, 340, 342-344, 346, 376, 378-380, 403-412, 434, 459.

The relevant residues for other members of the EGF receptor family can be determined from sequence alignments.

In an eighth aspect the present invention provides computer-assisted method for identifying potential compounds able to interact with a member of the EGF receptor family and thereby modulate an activity mediated by receptor, using a programmed computer comprising a processor, an input device, and an output device, comprising the steps of:

(a) inputting into the programmed computer, through the input device, data comprising the atomic coordinates of amino acids 1-501 of the EGF receptor molecule as shown in Appendix I, or structural coordinates having a root mean square deviation from the backbone atoms of said amino acids of not more than 1.5 Å, or one or more subsets of said amino acids, or one or more subsets of said amino acids related to the coordinates shown in Appendix I by whole body translations and/or rotations;

(b) generating, using computer methods, a set of atomic coordinates of a structure that possesses stereochemical complementarity to the atomic coordinates of amino acids 1-501 of the EGF receptor molecule as shown in Appendix 1, or structural coordinates having a root mean square deviation from the backbone atoms of said amino acids of not more than 1.5 Å, or one or more subsets of said amino acids, or one or more subsets of said amino acids related to the coordinates shown in Appendix I by whole body translations and/or rotations, thereby generating a criteria data set;

(c) comparing, using the processor, the criteria data set to a computer database of chemical structures;

(d) selecting from the database, using computer methods, chemical structures which are similar to a portion of said criteria data set; and

(e) outputting, to the output device, the selected chemical structures which are complementary to or similar to a portion of the criteria data set.

In a preferred embodiment of the eighth aspect the subset of amino acids are the amino acids (i) defining either or both the ligand binding surface(s), or (ii) defining dimerization interface.

In a further preferred embodiment the method is used to identify potential compounds which have the ability to decrease an activity mediated by the receptor.

In a further preferred embodiment of the eighth aspect, the method further comprises the step of selecting one or more chemical structures from step (e) which interact with a member of the EGF receptor family in a manner such as to interfere with the binding of natural ligand to:—

(i) one or more of the residues of EGFR selected from the group consisting of 11-18, 20, 22, 26, 29, 30, 45, 69, 89, 90, 98, 99, 101-103, 125, 127, 128, 325, 346, 348-350, 353-358, 382, 384, 408, 409, 411, 412, 415, 417, 418, 438, 440, 465 and 467 and combinations thereof; or

(ii) the corresponding region of other members of the EGF receptor family.

In a further preferred embodiment of the eighth aspect, the method further comprises the step of selecting one or more chemical structures from step (e) which interact with one or more of the residues of EGFR selected from the group consisting of amino acids 38, 86, 193-195, 204, 205, 229, 230, 239, 242-246, 248-253, 262-265, 275, 278-280, 282-288, 318 or the corresponding region of other members of the EGF receptor family.

In a further preferred embodiment of the eighth aspect, the method further comprises the step of obtaining a compound with a chemical structure selected in steps (d) and (e), and testing the compound for the ability to decrease an activity mediated by the receptor.

The present invention also provides a method of screening of a putative compound having the ability to modulate the activity of a molecule of the EGF receptor family, comprising the steps of identifying a putative compound by a method according to the first or third aspects, and testing the compound for the ability to increase or decrease an activity mediated by the molecule. In one embodiment, the test is carried out in vitro. Preferably, the in vitro test is a high throughput assay. In another embodiment, the test is carried out in vivo.

In a ninth aspect the present invention provides a computer for producing a three-dimensional representation of a molecule or molecular complex, wherein the computer comprises:

(a) a machine-readable data storage medium comprising a data storage material encoded with machine-readable data, wherein the machine readable data comprise the atomic coordinates of amino acids 1-501 of the EGF receptor molecule as shown in Appendix I, or structural coordinates having a root mean square deviation from the backbone atoms of said amino acids of not more than 1.5 Å, or one or more subsets of said amino acids, or one or more subsets of said amino acids related to the coordinates shown in Appendix I by whole body translations and/or rotations;

(b) a working memory for storing instructions for processing the machine-readable data;

(c) a central-processing unit coupled to the working memory and to the machine-readable data storage medium, for processing the machine-readable data into the three dimensional representation; and

(d) an output hardware coupled to the central processing unit, for receiving the three-dimensional representation.

In a preferred embodiment of the ninth aspect the subset of amino acids are the amino acids (i) defining either or both the ligand binding surface(s), or (ii) defining dimerization interface.

In a tenth aspect the present invention provides a compound able to interact with a member of the EGF receptor family and to modulate an activity mediated by the receptor, the compound being obtained by a method according to the present invention.

In a preferred embodiment of the tenth aspect, the compound is a mutant of the natural ligand of a receptor of the EGF receptor family, where at least one mutation occurs in the region of the natural ligand which interacts with the receptor.

In an eleventh aspect the present invention provides a compound which possesses stereochemical complementarity to a topographic region of a molecule of the EGF receptor family and modulates an activity mediated by the molecule, wherein the molecule is characterised by

(i) amino acids 1-501 of the EGF receptor positioned at atomic coordinates as shown in Appendix I, or structural coordinates having a root mean square deviation from the backbone atoms of said amino acids of not more than 1.5 Å;

(ii) one or more subsets of said amino acids related to the coordinates shown in Appendix I by whole body translations and/or rotations, or

(iii) amino acids present in the amino acid sequence of a member of the EGF receptor family, which form an equivalent three-dimensional structure to that of the receptor site defined by amino acids 1-501 of the EGF receptor positioned at atomic coordinates substantially as shown in Appendix I;

with the proviso that the compound is not a naturally occurring member of the EGF receptor family or a mutant thereof.

By “mutant” we mean a ligand which has been modified by one or more point mutations, insertions of amino acids or deletions of amino acids.

In one embodiment of the eleventh aspect, the topographic region of the molecule is defined by is the ligand binding surface defined by amino acids 11-18, 20, 22, 26, 29, 30, 45, 69, 89, 90, 98, 99, 101-103, 125, 127 and 128 and/or the ligand binding surface defined by amino acids 325, 346, 348-350, 353-358, 382, 384, 408, 409, 411, 412, 415, 417, 418, 438, 440, 465 and 467 or the corresponding regions of a member of the EGF receptor family.

In another embodiment of the eleventh aspect, the topographic region of the EGFR is defined by the dimerization interface defined by amino acids 38, 86, 193-195, 204, 205, 229, 230, 239, 242-246, 248-253, 262-265, 275, 278-280, 282-288, 318.

In preferred embodiments of the tenth and eleventh aspects, the stereochemical complementarity between the compound and the receptor is such that the compound has a Kd for the receptor site of less than 10⁻⁶M, more preferably less than 10⁻⁸M.

In other embodiments of the tenth and eleventh aspects, the compound decreases an activity mediated by the EGF receptor.

In a twelfth aspect, the present invention provides a pharmaceutical composition for preventing or treating a disease associated with signaling by a molecule of the EGF receptor family which comprises a compound according to the ninth or tenth aspects of the present invention and a pharmaceutically acceptable carrier or diluent.

In a thirteenth aspect the present invention provides a method of preventing or treating a disease associated with signaling by a molecule of the EGF receptor family which method comprises administering to a subject in need thereof a compound according to the ninth or tenth aspects of the present invention. Preferably, the disease is selected from psoriasis and tumour states comprising but not restricted to cancer of the breast, brain, colon, prostate, ovary, cervix, pancreas, lung, head and neck, and melanoma, rhabdomyosarcoma, mesothelioma, squamous carcinomas of the skin and glioblastoma.

In a fourteenth aspect, the present invention provides a method for evaluating the ability of a chemical entity to bind to EGFR, said method comprising the steps of:

(a) creating a computer model of at least one region of EGFR using structure coordinates wherein the root mean square deviation between said structure coordinates and the structure coordinates of amino acids 1-501 of EGFR as set forth in Appendix I or Appendix II is not more than about 1.5 Å;

(b) employing computational means to perform a fitting operation between the chemical entity and said computer model of the binding surface; and

(c) analysing the results of said fitting operation to quantify the association between the chemical entity and the binding surface model.

In one embodiment of the fourteenth aspect of the invention the region of EGFR is selected from the group consisting of the ligand binding surface defined by amino acids 11-18, 20, 22, 26, 29, 30, 45, 69, 89, 90, 98, 99, 101-103, 125, 127 and 128 and/or the ligand binding surface defined by amino acids 325, 346, 348-350, 353-358, 382, 384, 408, 409, 411, 412, 415, 417, 418, 438, 440, 465 and 467 348-350, 353-358, 382, 384, 408, 409, 411, 412, 415, 417, 418, 438 and 465 and a combination thereof.

In another embodiment of the fourteenth aspect the region of EGFR is the dimerization interface defined by amino acids 38, 86, 193-195, 204, 205, 229, 230, 239, 242-246, 248-253, 262-265, 275, 278-280, 282-288 and 318.

In a fifteenth aspect the present invention consists in a polypeptide complex in a crystallized form comprising the amino acids 1-501 of EGFR and TGFα.

It will be appreciated that isolated dimers of compounds comprising extracellular fragments of members of the EGF receptor family (e.g. dimers of fragment 1-501 of EGFR) in the back-to-back configuration may be useful therapeutic agents given their ability to compete with natural receptors for binding to ligands of the EGF receptor family.

Accordingly, in a sixteenth aspect the present invention provides a compound comprising fragment 1-501 of EGFR or an equivalent fragment of a member of the EGF receptor family, wherein the fragment is modified to induce dimerisation of the fragment in back-to-back configuration.

In one embodiment, the modification is made to a residue of the fragment which forms part of the back-to-back dimer interface. More preferably, the modification involves substitution of at least one residue which forms part of the back to back dimer with a cysteine residue. The substitution may be P248C and/or A265C. Alternatively, the substitution may be D279C.

In another embodiment of the sixteenth aspect, the modification involves insertion of a dimerization sequence into the fragment. A “dimerization” sequence allows the non-covalent association of one binding domain to another, with sufficient affinity to remain associated under normal physiological conditions.

Suitable dimerization domains that can be used in the context of the present invention would be known to those skilled in the art, or may be readily identified using standard methods such as the yeast two hybrid system and traditional biochemical affinity binding studies. For example, an in vivo library-versus-library selection of optimized protein-protein interactions is described in Pelletier et al., (1999) Nature Biotechnology 17, 683.

Suitable dimerization sequences may be derived, for example, from Jun and Fos, which are sequence specific DNA binding proteins that regulate transcription. Each protein has a bipartite DNA-binding domain consisting of an amphipathic helix that mediates dimerization through formation of a short coiled structure, termed a “leucine zipper”. Suitable dimerization pairs for use in the present invention may include the leucine zipper of Jun or Fos and a protein sequence that reacts with this leucine zipper. A method for identifying mammalian proteins that react with the leucine zipper of Jun is described in Chevray & Nathans, (1992) Proc. Natl. Acad. Sci. USA 89, 5789.

Suitable dimerization sequences for use in the present invention also include:

(i) Heterodimeric coiled-coil peptide pairs as described in Arndt et al., (2000) J. Mol. Biol. 295, 627;

(ii) The WW domain and ligands that bind thereto (see Dalby et al., (2000) Prot. Sci. 9, 2366);

(iii) The bacterial nucleoid-associated proteins H—NS and StpA which form homomeric or heteromeric complexes (see Dorman et al., (1999) Trends Microbiol. 7, 124); and

(iv) Antibody domains, such as the first constant domain (C_{H}1 and C_{L}) of an IgG1 (see, for example, Mueller et al., (1998) FEBS Lett 422, 259).

In one embodiment, the dimerization sequence is inserted between residues 194 and 195 or between residues 204 and 205 of EGFR or equivalent residues of another member of the EGF receptor family.

In yet another embodiment of the sixteenth aspect, the modification involves the lengthening of an appropriate loop structure (e.g. a loop within the S1 domain) which may then be cross-linked with the corresponding loop or a different loop of the dimer partner by a linker. The linker may be, for example, a disulphide bond. The lengthening of the loop may be achieved, for example, by the insertion of additional residues between residues 210 and 211 or between residues 297 and 298 of EGFR or the equivalent residues of another member of the EGF receptor family.

In another embodiment of the sixteenth aspect, the fragment is conjugated to a molecule. The molecule may be, for example, a constant domain of an immunoglobulin molecule.

The present invention also encompasses compounds of the sixteenth aspect in dimer form.

The information provided in Appendix I and II also shows that there are a number of loop structures in the EGFR. From the three dimensional structure antibodies directed against these would interfere with binding of the natural ligand to the receptor or with the formation of active dimers.

Accordingly in a seventeenth aspect the present invention consists in an antibody which binds to EGFR, the antibody being directed against (i) EGFR residues 100-108, 315-327 or 353-362; or (ii) EGFR residues 190-207, 240-305 or parts thereof or the corresponding regions of a member of the EGF receptor family.

Antibodies of the present invention may be produced, for example, by immunizing mice with purified EGFR fragment 1-501. After determining that the mice are producing anti-EGFR antibodies, hybridomas may be prepared and antibody specificity assayed by ELISA or Flow Cytometry using two cell lines: Baf/wt-EGFR cells and Baf/EGFR-“mutation x” cells. These mouse cell lines express either the wild type EGFR or the EGFR containing an Ala substitution (ie mutation x) within the specific site against which the antibody is to be directed. When hybridomas secreting antibodies which recognize Baf/wt-EGFR, but not Baf/EGFR-“mutant x” are identified, the corresponding hybridoma may be cloned and the monoclonal antibody purified.

Alternatively, in raising antibodies of the invention, it may be desirable to use derivatives of the peptides or loop structures which are conformationally constrained. Conformational constraint refers to the stability and preferred conformation of the three-dimensional shape assumed by a peptide. Conformational constraints include local constraints, involving restricting the conformational mobility of a single residue in a peptide; regional constraints, involving restricting the conformational mobility of a group of residues, which residues may form some secondary structural unit; and global constraints, involving the entire peptide structure.

The active conformation of the peptide may be stabilized by a covalent modification, such as cyclization or by incorporation of gamma-lactam or other types of bridges. For example, side chains can be cyclized to the backbone so as create a L-gamma-lactam moiety on each side of the interaction site. See, generally, Hruby et al., “Applications of Synthetic Peptides,” in Synthetic Peptides: A User's Guide: 259-345 (W. H. Freeman & Co. 1992). Cyclization also can be achieved, for example, by formation of cystine bridges, coupling of amino and carboxy terminal groups of respective terminal amino acids, or coupling of the amino group of a Lys residue or a related homolog with a carboxy group of Asp, Glu or a related homolog. Coupling of the alpha-amino group of a polypeptide with the epsilon-amino group of a lysine residue, using iodoacetic anhydride, can be also undertaken. See Wood and Wetzel, 1992, Int'l J. Peptide Protein Res. 39: 533-39.

Further the conformation of the peptide analogues may be stabilised by including amino acids modified at the alpha carbon atom (eg. α-amino-150-butyric acid) (Burgess and Leach, 1973, Biopolymers 12(12):2691-2712; Burgess and Leach, 1973, Biopolymers 12(11):2599-2605) or amino acids which lead to modifications on the peptide nitrogen atom (eg. sarcosine or N-methylalanine) (O'Donohue et al, 1995, Protein Sci. 4(10):2191-2202).

Another approach described in U.S. Pat. No. 5,891,418 is to include a metal-ion complexing backbone in the peptide structure. Typically, the preferred metal-peptide backbone is based on the requisite number of particular coordinating groups required by the coordination sphere of a given complexing metal ion. In general, most of the metal ions that may prove useful have a coordination number of four to six. The nature of the coordinating groups in the peptide chain includes nitrogen atoms with amine, amide, imidazole, or guanidino functionalities; sulfur atoms of thiols or disulfides; and oxygen atoms of hydroxy, phenolic, carbonyl, or carboxyl functionalities. In addition, the peptide chain or individual amino acids can be chemically altered to include a coordinating group, such as for example oxime, hydrazino, sulfhydryl, phosphate, cyano, pyridino, piperidino, or morpholino. The peptide construct can be either linear or cyclic, however a linear construct is typically preferred.

As will be readily understood by person skilled in this field the methods of the present invention provide a rational method for designing and selecting compounds including antibodies which interact with members of the EGF receptor family. In the majority of cases these compounds will require further development in order to increase activity. Such further development is routine in this field and will be assisted by the structural information provided in this application. It is intended that in particular embodiments the methods of the present invention includes such further developmental steps.

In yet a further, eighteenth, aspect, the invention provides a method of utilizing molecular replacement to obtain structural information about a molecule or a molecular complex of unknown structure, comprising the steps of:

(i) crystallising said molecule or molecular complex;

(ii) generating an X-ray diffraction pattern from said crystallized molecule or molecular complex;

(iii) applying at least a portion of the structure coordinates set forth in Appendix I or Appendix II to the X-ray diffraction pattern to generate a three-dimensional electron density map of at least a portion of the molecule or molecular complex whose structure is unknown.

The term “molecular replacement” refers to a method that involves generating a preliminary model of an EGF receptor family member extracellular domain crystal whose structure coordinates are unknown, by orienting and positioning a molecule whose structure coordinates are known (e.g., EGFR 1-501 coordinates from Appendix I or Appendix II) within the unit cell of the unknown crystal so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal (Lattman, 1985, Methods in Enzymology 115: 55-77; M. G. Rossmann, ed., “The Molecular Replacement Method”, Int. Sci. Rev. Ser., No. 13, Gordon & Breach, New York, 1972). Using the structure coordinates of the EGFR 1-501 provided by this invention, molecular replacement may be used to determine the structural coordinates of a member of the EGF receptor family.

Throughout this specification, the terms “S1” domain and “cys-rich 1” (“CR1”) domain are used interchangeably. Similarly, the terms “S2” domain and “cys-rich 2” (“CR2”) domain are used interchangeably.

Throughout this specification, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF THE FIGURES

FIG. 1: Structure-based sequence alignment of the EGFR residues 1-501 and corresponding residues of ErbB-2, ErbB-3 and ErbB-4.

FIG. 2: Sequence alignment of EGF-like domains of ligands of the EGFR family. Note that the start and end of some of these domains are not precisely defined. The sequences are for the human forms of the proteins except for epigen and the EGF-like domain in neuregulin-4 which are the mouse forms of the respective proteins. Abbreviations: EGF—epidermal growth factor; TGF-α—transforming growth factor alpha; HB-EGF—heparin binding epidermal growth factor; NRG—neuregulin. There are four known neuregulin genes (NRG1, NRG2, NRG3 and NRG4), some of which encode alternatively spliced forms of the EGF-like domain. These forms are identified as the α- or β-form of the EGF-like domain.

FIG. 3. Polypeptide trace for the structure of the 2:2 complex of sEGFR501 and TGFα back-to-back dimer, comprising receptor molecule A, receptor molecule B, TGFα molecule C and TGFα molecule D. The dimer axis lies vertically, in the page.

FIG. 4. Structure-based sequence alignment of the human EGFR ectodomain, human TGFα and related proteins. (A) The receptor L1 and L2 domains plus the first module of the cys rich regions, S1 and S2. (B) Modules 2 to 8 of the receptor cys rich region S1 and modules 2 to 7 of S2. (C) Human TGFα, EGF and heparin binding EGF. Numbers in parentheses show where amino acid have been omitted and positions with conserved physicochemical properties of amino acids are boxed. Secondary structure elements are indicated above the sequences (and below in A), with shading as in FIG. 5A. Also indicated are disulfide bonds and residues buried at protein-protein interfaces: L1-TGFα, 1; L2-TGFα, 2; L1-L2 contacts, 3 in A; L1-& L2-TGFα, 3 in B; S1 loop, L; residues to which the S1 loop binds, P; other residues in the dimer interface, D. Three types of disulfide bonded modules are indicated by bars below the sequences and residues not conforming to the S1 pattern are shaded grey.

FIG. 5. Comparison of sEGFR501 with the first three domains of IGF-1R. Domains 1-3 of IGF-1R are on the left, sEGFR501 as it appears in the complex is on the right. For clarity the ligand in the TGFα:sEGFR501 complex is not shown. L1 domains are oriented similarly.

FIG. 6. Structure of the ligand:receptor binding surfaces. Ribbon representation showing the contacts between sEGFR501 and TGFα viewed from the left in FIG. 3. Residue numbers for two important residues in TGFα are below the side chains.

FIG. 7. Stereoview of the molecule A S1 loop contacts with S1 of molecule B in the back-to-back dimer interface. Inter-chain hydrogen-bonds are drawn in black along with the hydrogen-bond from AsnA247 which stabilises the loop tip conformation. The single letter code and residue number is used for amino acid residues. The dimer axis lies vertically at the left between H280.

FIG. 8: Functional characterization of EGFR mutants expressed in BaF/3 cells. (A) Ligand binding by wild type and mutant EGFRs expressed in BaF/3 cells. Scatchard plots of 1251-EGF binding to clones expressing the wt, E21A or ΔCR1EGFR were analyzed using the Radlig program to yield estimates of receptor affinity. The three cell lines expressed comparable receptor numbers as assessed by M2 or 528 antibody binding and FACS analysis. Shown are the plots for cold ligand titration assay; identical results were obtained titrating the radiolabelled EGF (hot titration). (B) EGF-dependent tyrosine kinase activation. This was determined in total cell lysates by sequential immunoblotting with anti-phosphotyrosine (top) or anti-EGFR (bottom) antibodies. The anti-EGFR antibodies have slightly lower affinity for the hyperphosphorylated form of the EGFR. The results are representative of multiple experiments on at least four independently derived clones for each mutant. (C) Ligand-induced EGFR dimerization. Cross-linking of the EGFR via the extracellular portion was performed at 37° C. to maximize dimer yield. Samples were analyzed by SDS-PAGE on 3-8% gradient gels and immunoblotting with anti-EGFR antibodies. These data are representative of at least four separate experiments. (D) Ligand-induced sEGFR501 dimerization. Cross-linking of wild type and CR1 loop mutant (Tyr246Asp, Asn247Ala, Thr249Asp, Tyr251Glu, Gln252Ala and Met253Asp) was carried out as described previously (Elleman et al., 2001. Biochemistry 40:8930-8939).

### KEY TO SEQUENCE LISTING

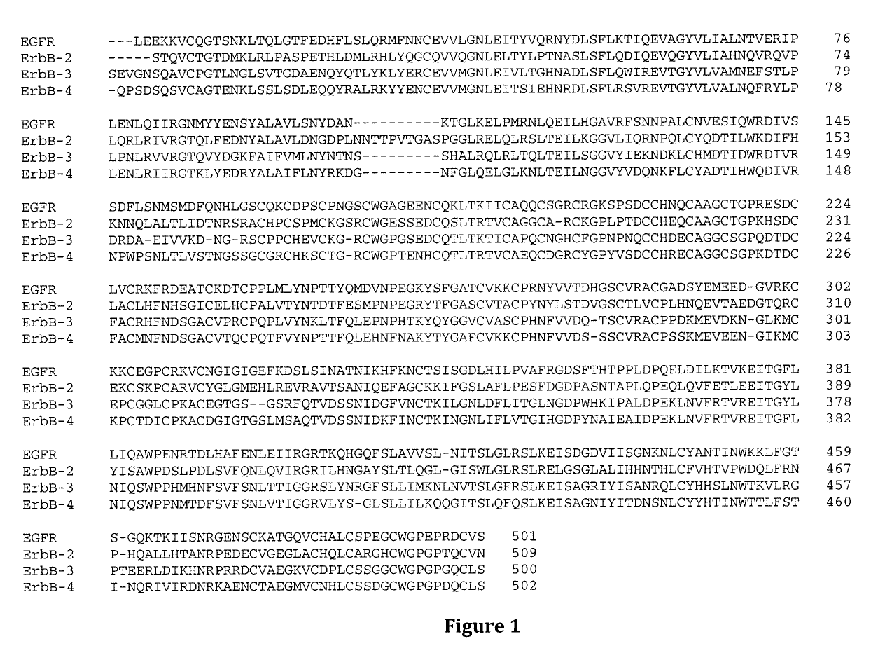

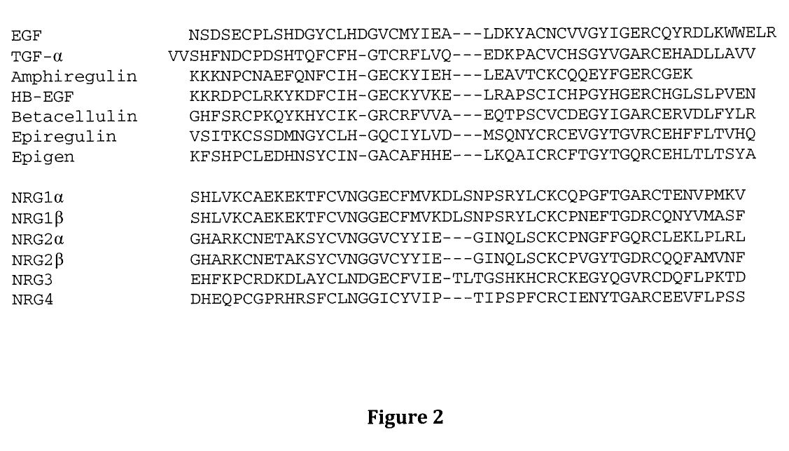

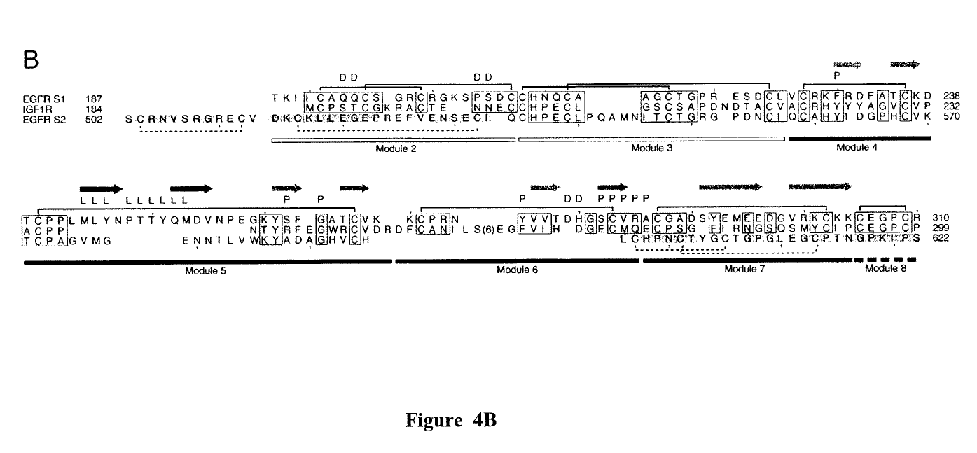

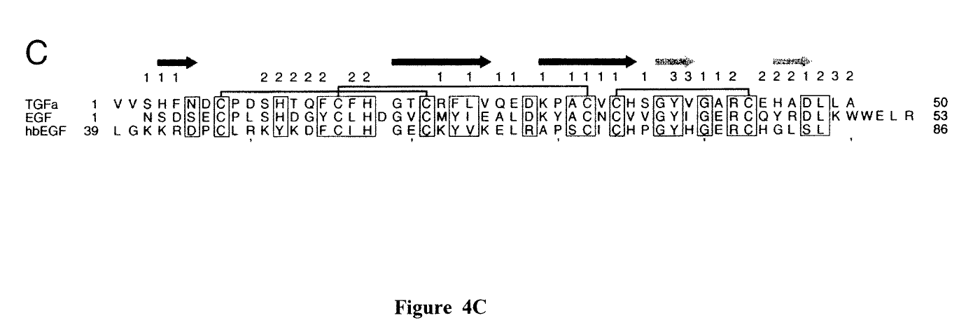

SEQ ID NO:1: EGFR as shown in FIG. 1
SEQ ID NO:2: ErbB-2 as shown in FIG. 1
SEQ ID NO:3: ErbB-3 as shown in FIG. 1
SEQ ID NO:4: ErbB-4 as shown in FIG. 1
SEQ ID NO:5: EGF domain as shown in FIG. 2
SEQ ID NO:6: TGF-α domain as shown in FIG. 2
SEQ ID NO:7: Amphiregulin domain as shown in FIG. 2
SEQ ID NO:8: HB-EGF domain as shown in FIG. 2
SEQ ID NO:9: Betacellulin domain as shown in FIG. 2
SEQ ID NO:10: Epiregulin domain as shown in FIG. 2
SEQ ID NO:11: Epigen domain as shown in FIG. 2
SEQ ID NO:12: NRG1α domain as shown in FIG. 2
SEQ ID NO:13: NRG1β domain as shown in FIG. 2
SEQ ID NO:14: NRG2α domain as shown in FIG. 2
SEQ ID NO:15: NRG2β domain as shown in FIG. 2
SEQ ID NO:16: NRG3 domain as shown in FIG. 2
SEQ ID NO:17: NRG4 domain as shown in FIG. 2
SEQ ID NO:18: EGFR L1 domain as shown in FIG. 4A
SEQ ID NO:19: IGF 1R L1 domain as shown in FIG. 4A
SEQ ID NO:20: IGF 1R L2 domain as shown in FIG. 4A
SEQ ID NO:21: EGFR L2 domain as shown in FIG. 4A
SEQ ID NO:22: EGFR S1 domain as shown in FIG. 4B
SEQ ID NO:23: IGF 1R S1 domain as shown in FIG. 4B
SEQ ID NO:24: EGFR S2 domain as shown in FIG. 4B
SEQ ID NO:25: TGFα domain as shown in FIG. 4C
SEQ ID NO:26: EGF domain as shown in FIG. 4C
SEQ ID NO:27: hbEGF domain as shown in FIG. 4C

### DETAILED DESCRIPTION OF PREFERRED EMBODIMENTS OF THE INVENTION

The present inventors have now obtained three dimensional structural information about the EGF receptor which enables a more accurate understanding of how the binding of ligand leads to signal transduction. Such information provides a rational basis for the development of ligands for specific therapeutic applications, something that heretofore could not have been predicted de novo from available sequence data.

The precise mechanisms underlying the binding of agonists and antagonists to the EGF receptor are not fully clarified. However, the binding of ligands to the receptor site, preferably with an affinity in the order of 10⁻⁸M or higher, is understood to arise from enhanced stereochemical complementarity relative to naturally occurring EGF receptor ligands.

Such stereochemical complementarity, pursuant to the present invention, is characteristic of a molecule that matches intra-site surface residues lining the groove of the receptor site as enumerated by the coordinates set out in Appendix I or Appendix II. Appendix II is a refined version of the coordinates provided in Appendix I.

Substances which are complementary to the shape and electrostatics or chemistry of the receptor site characterised by amino acids positioned at atomic coordinates set out in Appendix I or Appendix II will be able to bind to the receptor, and when the binding is sufficiently strong, substantially prohibit binding of the naturally occurring ligands to the site.

It will be appreciated that it is not necessary that the complementarity between ligands and the receptor site extend over all residues lining the groove in order to inhibit binding of the natural ligand.

In general, the design of a molecule possessing stereochemical complementarity can be accomplished by means of techniques that optimize, chemically and/or geometrically, the “fit” between a molecule and a target receptor. Known techniques of this sort are reviewed by Sheridan and Venkataraghavan, Acc. Chem. Res. 1987 20 322; Goodford, J. Med. Chem. 1984 27 557; Beddell, Chem. Soc. Reviews 1985, 279; Hol, Angew. Chem. 1986 25 767, Verlinde C. L. M. J & Hol, W. G. J. Structure 1994, 2, 577, Walters, W. P., Stahl, M. T., Murcko, M. A., Drug Discovery Today 1998, 3, 160; Langer, T. and Hoffmann, R. D., Current Pharmaceutical Design 2001, 7, 509; Good, A., Current Opinion in Drug Disc. Devel. 2001, 5, 301; and Gane, P. J. and Dean, P. M., Curr. Opinion Struct. Biol., 2000, 10, 401, the respective contents of which are hereby incorporated by reference. See also Blundell et al., Nature 1987 326 347 (drug development based on information regarding receptor structure) and Loughney, D. A., Murray, W. V., and Jolliffe, L. K. Med. Chem. Res. 1999, 9, 579 (database mining application on the growth hormone receptor).

There are two preferred approaches to designing a molecule, according to the present invention, that complements the stereochemistry of the EGF receptor. The first approach is to in silicon directly dock molecules from a three-dimensional structural database, to the receptor site, using mostly, but not exclusively, geometric criteria to assess the goodness-of-fit of a particular molecule to the site. In this approach, the number of internal degrees of freedom (and the corresponding local minima in the molecular conformation space) is reduced by considering only the geometric (hard-sphere) interactions of two rigid bodies, where one body (the active site) contains “pockets” or “grooves” that form binding sites for the second body (the complementing molecule, as ligand).

This approach is illustrated by Kuntz et al., J. Mol. Biol. 1982 161 269, and Ewing, T. J. A. et al., J. Comput-Aid. Mol. Design. 2001, 15, 411, the contents of which are hereby incorporated by reference, whose algorithm for ligand design is implemented in a commercial software package, DOCK version 4.0, distributed by the Regents of the University of California and further described in a document, provided by the distributor, which is entitled “Overview of the DOCK program suite” the contents of which are hereby incorporated by reference. Pursuant to the Kuntz algorithm, the shape of the cavity represented by the EGF receptor site is defined as a series of overlapping spheres of different radii. One or more extant databases of crystallographic data, such as the Cambridge Structural Database System maintained by Cambridge University (University Chemical Laboratory, Lensfield Road, Cambridge CB2 1EW, U.K.), the Protein Data Bank maintained by the Research Collaboratory for Structural Bioinformatics (Rutgers University, N.J., U.S.A.), LeadQuest (Tripos Associates, Inc., St. Louis, Mo.), Available Chemicals Directory (Molecular Design Ltd., San Leandro, Calif.), and the NCI database (National Cancer Institute, U.S.A) is then searched for molecules which approximate the shape thus defined.

Molecules identified in this way, on the basis of geometric parameters, can then be modified to satisfy criteria associated with chemical complementarity, such as hydrogen bonding, ionic interactions and Van der Waals interactions. Different scoring functions can be employed to rank and select the best molecule from a database. See for example Bohm, H.-J. and Stahl, M. Med. Chem. Res. 1999, 9, 445. The software package FlexX, marketed by Tripos Associates, Inc. (St. Louis, Mo.) is another program that can be used in this direct docking approach (see Rarey, M. et al., J. Mol. Biol. 1996, 261, 470).

The second preferred approach entails an assessment of the interaction of respective chemical groups (“probes”) with the active site at sample positions within and around the site, resulting in an array of energy values from which three-dimensional contour surfaces at selected energy levels can be generated. The chemical-probe approach to ligand design is described, for example, by Goodford, J. Med. Chem. 1985 28 849, the contents of which are hereby incorporated by reference, and is implemented in several commercial software packages, such as GRID (product of Molecular Discovery Ltd., West Way House, Elms Parade, Oxford OX2 9LL, U.K.). Pursuant to this approach, the chemical prerequisites for a site-complementing molecule are identified at the outset, by probing the active site with different chemical probes, e.g., water, a methyl group, an amine nitrogen, a carboxyl oxygen, and a hydroxyl. Favored sites for interaction between the active site and each probe are thus determined, and from the resulting three-dimensional pattern of such sites a putative complementary molecule can be generated. This may be done either by programs that can search three-dimensional databases to identify molecules incorporating desired pharmacophore patterns or by programs which using the favored sites and probes as input perform de novo design.

Programs suitable for searching three-dimensional databases to identify molecules bearing a desired pharmacophore include: MACCS-3D and ISIS/3D (Molecular Design Ltd., San Leandro, Calif.), ChemDBS-3D (Chemical Design Ltd., Oxford, U.K.), and Sybyl/3 DB Unity (Tripos Associates, Inc., St. Louis, Mo.).

Programs suitable for pharmacophore selection and design include: DISCO (Abbott Laboratories, Abbott Park, Ill.), Catalyst (Accelrys, San Diego, Calif.), and ChemDBS-3D (Chemical Design Ltd., Oxford, U.K.).

Databases of chemical structures are available from a number of sources including Cambridge Crystallographic Data Centre (Cambridge, U.K.), Molecular Design, Ltd., (San Leandro, Calif.), Tripos Associates, Inc. (St. Louis, Mo.), and Chemical Abstracts Service (Columbus, Ohio).

De novo design programs include Ludi (Biosym Technologies Inc., San Diego, Calif.), Leapfrog (Tripos Associates, Inc.), Aladdin (Daylight Chemical Information Systems, Irvine, Calif.), and LigBuilder (Peking University, China).

Those skilled in the art will recognize that the design of a mimetic may require slight structural alteration or adjustment of a chemical structure designed or identified using the methods of the invention.

The invention may be implemented in hardware or software, or a combination of both. However, preferably, the invention is implemented in computer programs executing on programmable computers each comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program is preferably implemented in a high level procedural or object-oriented programming language to communicate with a computer system. However, the programs can be implemented in assembly or machine language, if desired. In any case, the language may be compiled or interpreted language.

Each such computer program is preferably stored on a storage medium or device (e.g., ROM or magnetic diskette) readable by a general or special purpose programmable computer, for configuring and operating the computer when the storage media or device is read by the computer to perform the procedures described herein. The inventive system may also be considered to be implemented as a computer-readable storage medium, configured with a computer program, where the storage medium so configured causes a computer to operate in a specific and predefined manner to perform the functions described herein.

Compounds designed according to the methods of the present invention may be assessed by a number of in vitro and in vivo assays of hormone function. For example, the identification of EGF receptor antagonists of may be undertaken using a solid-phase receptor binding assay. Potential antagonists may be screened for their ability to inhibit the binding of europium-labelled EGF receptor ligands to soluble, recombinant EGF receptor in a microplate-based format. Europium is a lanthanide fluorophore, the presence of which can be measured using time-resolved fluorometry. The sensitivity of this assay matches that achieved by radioisotopes, measurement is rapid and is performed in a microplate format to allow high-sample throughput, and the approach is gaining wide acceptance as the method of choice in the development of screens for receptor agonists/antagonists (see Apell et. al. J. Biomolec. Screening 3:19-27, 1998: Inglese et. al. Biochemistry 37:2372-2377, 1998).

Binding affinity and inhibitor potency may be measured for candidate inhibitors using biosensor technology.

The EGF receptor antagonists may be tested for their ability to modulate receptor activity using a cell-based assay incorporating a stably transfected, EGF-responsive reporter gene (Souriau et al., 1997, Nucleic Acids Res. 25:1585-1590). The assay addresses the ability of EGF to activate the reporter gene in the presence of novel ligands. It offers a rapid (results within 6-8 hours of hormone exposure), high-throughput (assay can be conducted in a 96-well format for automated counting) analysis using an extremely sensitive detection system (chemiluminescence). Once candidate compounds have been identified, their ability to antagonise signal transduction via the EGF-R can be assessed using a number of routine in vitro cellular assays such as inhibition of EGF-mediated cell proliferation. Ultimately, the efficiency of antagonist as a tumour therapeutic may be tested in vitro in animals beating tumour isografts and xenografts as described (Rockwell et al., 1997, Proc Natl Acad Sci USA 94:6523-6528; Prewett et al., 1998 Clin Cancer Res 4:2957-2966).

Tumour growth inhibition assays may be designed around a nude mouse xenograft model using a range of cell lines. The effects of the receptor antagonists and inhibitors may be tested on the growth of subcutaneous tumours.

### EXAMPLES

### Example 1

### Protein Preparation of sEGFR501

The derivation of stably transfected Lec8 cells expressing sEGFR501 and the subsequent purification and characterisation of the secreted ectodomain has been described in detail (Elleman et al., 2001, Biochemistry 40:8930-8939.). Purified sEGFR501 was shown, by isoelectric focusing gels to be unstable on storage, the majority of isoforms being transformed into products with less acidic isoelectric points. This change was accompanied by a small mobility increase (estimated at 1-2 kDa) on SDS polyacrylamide gels. N-terminal sequence analysis showed that the new product retained the expressed N-terminus of sEGFR501, suggesting that the apparent 1-2 kDa reduction in mass and increase in positive charge might be due to partial or complete loss of the acidic-residue rich C-terminal tag and enterokinase cleavage site. Prolonged storage led to the majority of protein converting to the least acidic isoform of pI˜6.6, which appeared to remain stable. The conversion of a fresh preparation of sEGFR501 to a stable, less acidic isoform was more reproducible and rapid if it was subject to limited proteolysis at ambient temperature in Tris-buffered saline (pH8) for ˜180 min with endoproteinase Asp-N (Boehringer-Mannheim) at an enzyme:protein ratio of 1:1000 (w/w). The least-acidic isoform of apparent pI˜6.2 was isolated from the other components by anion exchange chromatography. The digest was bound to three Uno Q2 columns (BioRad) connected in series to a BioLogic HR liquid chromatography instrument in 20 mM ethanolamine/50 mM taurine pH8.0 buffer and the least acidic form was the first product obtained by isocratic elution in the same buffer containing 15 mM lithium acetate. The purified protein was incubated with endoglycosidase F (PNGase-free-Boehringer Mannheim) at a ratio of 10-20 Units/mg protein, followed by rechromatography over Superdex 200 to remove enzyme and low molecular weight cleavage products.

### Example 2

### Crystallization and Data Collection

sEGFR501 obtained from the above procedures appeared nearly homogeneous on SDS and IEF gels and was used in crystallization trials alone and in combination with several ligands. The best diffracting crystals were obtained from mixtures containing a five-fold molar quantity of human TGFα (GroPep receptor grade) compared to sEGFR501. Crystals of sEGFR501 in complex with TGFα were grown in 7% PEG 3350, 20% Trehalose, 10 mM CdCl₂ and 100 mM HEPES, pH 7.5, and belonged to the space group P21 (a=51.59, b=198.71, c=78.90 Å, β=102.03°). These crystals were cryo-cooled to −170° C. in the same mother liquor. Data were recorded on a Rigaku RAXIS VI area detector using a Siemens M18XHF X-ray generator with Yale/MSC mirrors or a Rigaku RU300 generator and AXCO capillary optics. Crystals were also derivatised by soaking in mother liquor containing 1-10 mM heavy atom compounds and diffractions data were collected as before and statistics are given in Table 1. The resolution limit was defined as where I/σ=2 for 50% of the reflections. Notable anisotropy was observed for the diffraction limit of the crystals and in the mosaic spread of diffraction maxima.

### Example 3

### Phase Determination and Structure Refinement

Phasing by multiple isomorphic replacement was performed with programs from CCP4 (Collaborative Computational Project Number 4, 1994) and SHARP (De La Fortelle and Bricogne, 1996, Methods Enzymol. 276: 472-494) and the resulting electron density maps were improved by solvent flattening and histogram matching with DM (Cowtan, K. 1994, Joint CCP4 and ESF-EACBM Newslett. Protein Crystallogr. 31:34-38). Details are given in Table 1. Density averaging using noncrystallographic symmetry was not of much value as the proteins corresponded to more than three rigid groups. The polypeptide chains for two receptor and two ligand molecules were fitted manually and refined with CNS (Brunger, et al., 1998, X-PLOR Reference Manual 3.851, Yale Univ., New Haven, Conn.). As the highest resolution data were collected for the PIP derivative these data were use for the final stages of refinement. During the refinement an overall anisotropic temperature factor was applied, with the magnitude of the semi-axes being −18.4, 5.6 and 12.7 Å². The refined structure contains 1097 amino acids, 14 carbohydrate residues, 7 Pt²⁺, 11 Cd²⁺ and 4 Cl⁻ ions and 79 water molecules. Poor density was observed for residues 148-160 and 289-307 in each receptor and no density was found for ligand residues C1 and D1-D2 and receptor residues A306 and beyond residues A500 and B501.

### Example 4

### Construction of N-Terminal Tagged EGF Receptor and Mutants

The polymerase chain reaction (PCR) using a human EGFR cDNA (Accession # x00588) (15) Ullrich et al., 1984, Nature 309:418-425) was used to generate EGFR expression constructs. It is noted that the original EGFR cDNA sequence contains an error at position 1806G (Accession # x00588). The correct base is 1806C, which destroys the Hind III restriction site in the original cDNA sequence. To construct the FLAG tag at the N-terminus of the receptor, PCR products containing EGFR leader sequence (and small portion of 5′ non-coding sequence, base pair 131 to 261), followed by the FLAG coding sequences with Hind III and Xho I on its 5′ and 3′ ends, respectively, were generated and cloned into a mammalian expression vector pcDNA3 (Invitrogen) using those restriction sites. The Xho I site coding for Leu and Glu of mature EGFR residues 1 and 2 was generated by silent mutation and an Xba I site was generated after the stop codon (3817-3819) of EGFR cDNA using PCR. Cloning such modified EGFR cDNA into the FLAG tag containing pcDNA3 vector yielded the wild-type N-terminus tagged EGF receptor construct, M2-EGFR. PCR products containing point mutations and S1-loop deletion were cloned using the wild-type M2-EGFR as a template. The point mutation constructs are E21A, R470L, N473D, S474E and A477D. The S1-loop deletion construct contains a replacement of nucleotides 988-1035 by GCC, resulting in S1-loop residues 244-259 being replaced by a single alanine residue. The sEGFR501S1-loop mutant (Tyr246Asp, Asn247Ala, Thr249Asp, Tyr251 Glu, Gln252Ala and Met253Asp) was generated by oligonucleotide-directed in vitro mutagenesis using the USB-T7 Gen kit, transiently expressed, purified and characterised as described previously (Elleman et al., 2001. Biochemistry 40:8930-8939).

### Example 5

### Transient Expression of Wild-Type and Mutant EFGR

NIH3T3 and 293 cells were obtained from the American Type Culture Collection. The cells were grown in a 10% CO₂ atmosphere at 37° C. in Dulbecco's modified Eagle's medium (for NIH3T3) or in RPMI medium (for 293) (both from Life Technologies. Inc.) containing 10% foetal bovine serum (CSL, Australia), 60 μg/ml penicillin and 100 μg/ml streptomycin. Transient transfections were performed using FuGENETM 6 (Roche Molecular Biochemicals) according to manufacture's protocol. Cells were seeded at ˜10% (for NIH3T3) or ˜25% (for 293) confluency in 6-well plate and transfected with 0.5 μg plasmid DNA per construct per well. Transfected cells were assayed two days later. For western blotting, cells were washed with serum-free medium, starved for 2 hr and treated with or without EGF (100 ng/ml) for 10 min. Whole cell lysates were prepared, fractionated by SDS-gel electrophoresis using 4-20% polyacrylamide gels and western blotted using the monoclonal antibodies M2 (anti-FLAG, Sigma) and 4G10 (anti-phosphotyrosine, Upstate Biotechnology) as described (Walker et al, 1998, Growth Factors 16, 53-67).

### Example 6

### Characterisation of Wild-Type and Mutant EGFR Stably Expressed in BaF/3 Cells

The isolation and characterisation of stably transfected cell lines expressing wild-type and mutant EGFRs was performed using the I13-dependent murine hemopoietic lineage BaF/3 (Walker et al, 1998, Growth Factors 16, 53-67). Expression vectors containing the appropriate EGFR constructs were transfected individually by electroporation using a Gene Pulser (BioRad) according to manufacturer's instructions. Neomycin-resistant pools were generated by selection in G418, and cloned by limiting dilution to obtain stable cell lines. Cell-surface expression of receptors was detected by FACScan (Fluorescence Activated Cell Scan, Becton and Dickinson) using the anti-EGFR monoclonal antibody 528 (Gill et al., 1984, J. Biol. Chem. 259:7755-7760) and the M2 anti-FLAG antibody (Brizzard et al., 1994, Biotechniques 16:730-735). Ligand binding studies and Scatchard analysis were performed using iodinated murine EGF as previously described (Walker et al, 1998, Growth Factors 16, 53-67). Scatchard plots and estimates of affinities and receptor numbers were obtained using the Radlig program (Kell for Windows, BioSoft). Ligand-induced receptor kinase activation was analysed by immunoblotting cell lysates with 4G10. For receptor cross-linking studies, washed cells were incubated in PBS with or without EGF (100 ng/ml) and with or without BS3 (Pierce; 1.3 mM) for 20 min at 37° C. The cells were then lysed and analysed by immunoblotting using a polyclonal sheep anti-EGFR antibody (Upstate Biotechnology) as described (Walker et al., 1998. Mol. Cell. Biol. 18:7192-7204).

### Example 7

### Overall Structure

sEGFR501 is comprised of three structural domains, namely L1, S1 and L2 plus the first module from the second cys-rich region S2. Crystals of TGFα:sEGFR501 contain two molecules of each polypeptide in the asymmetric unit. There are two possible dimer interactions: a back-to-back dimer dominated by interactions between the S1 domains of each receptor and a head-to-head dimer involving contacts between the L1 and L2 domains. The back-to-back complex is approximately 33×78×103 Å while the head-to-head complex is 65×75×128 Å. Each TGFα molecule is clamped between the L1 and L2 domains from the same sEGFR501 molecule, and makes contact with only one receptor molecule in the dimer. In the back-to-back dimer the two ligands are located on opposite sides of the complex with the closest approach 70.9 Å apart. In the head-to-head dimer the two ligands are centrally located, and are separated by 15 Å.

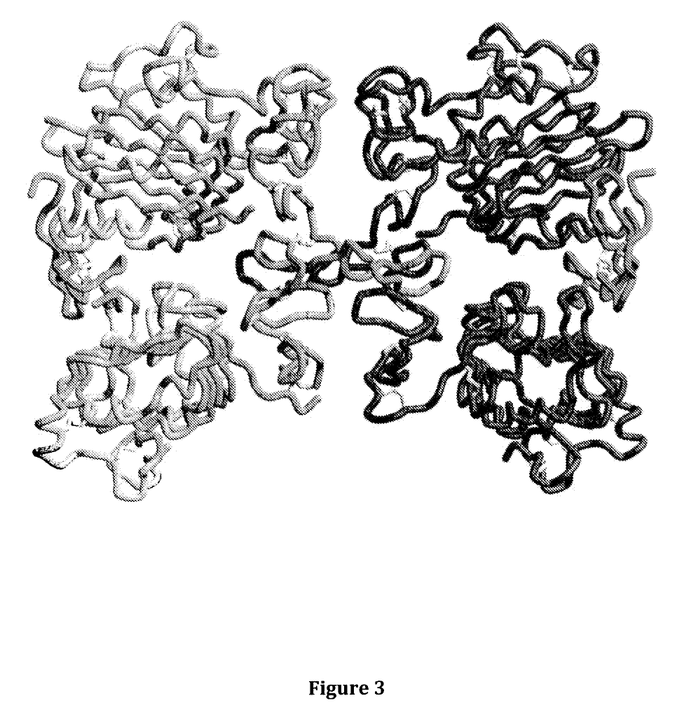

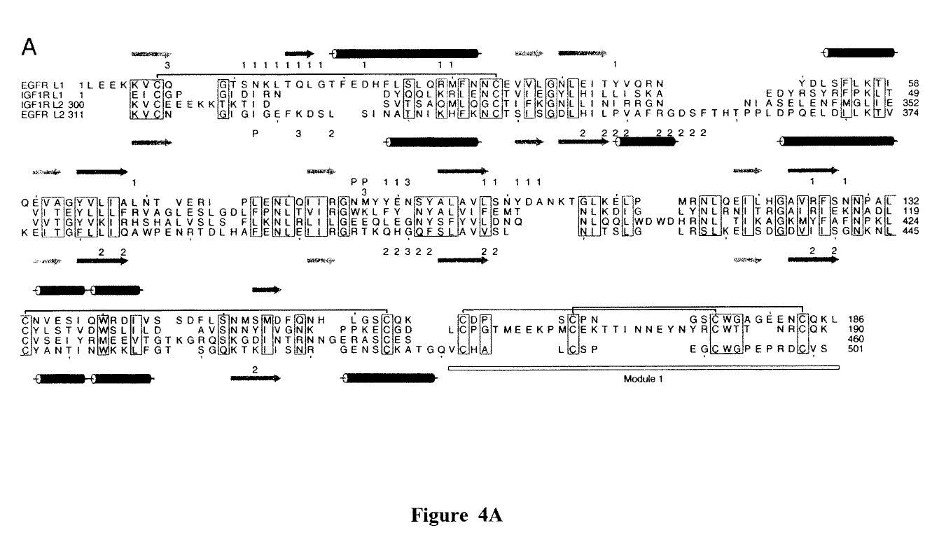

We conclude that the back-to-back dimer corresponds to the 2:2 TGFα:sEGFR501 complex that is formed in solution (Elleman et al., 2001. Biochemistry 40:8930-8939) from comparisons of the amount of buried surface area in the two dimer options, the lack of symmetry in the head-to-head dimer compared to that seen in the back-to-back dimer, the sequence conservation at the dimer interfaces (described later) and the characteristics of the receptors mutated at both interfaces (described later). In the head-to-head dimer only 510 Å2 of accessible surface area is buried on each molecule and this is distributed over two patches 39 Å apart. The residues involved are 21, 24, 25, 28 and 48-51 on both L1s, 471, 473, 474, 476 and 477 on both L2s plus 32 (molecule A) and 443 and 478 from molecule B. In contrast, in the back-to-back dimer 1125 Å2 on each receptor is buried. Biologically relevant protein-protein interfaces usually bury more than 700 Å2 of surface per molecule and often about 1000 Å2 (Lo Conte et al., 1999, J. Mol. Biol. 285:2177-2198), implying that the back-to-back configuration is more likely to be the functional dimer. There is a lack of symmetry at the two L1-L2′ interfaces in the head-to-head dimer which corresponds to a 6 Å translation of the L2′ helix (residues 471-479) relative to the L1 helix. Such structural ambiguity is not seen in the back-to-back dimer (FIG. 3), the non-crystallographic symmetry being very close to a pure two-fold rotation, implying that this is the functional dimer. It is further supported by experiments where a model of the EGF receptor S2 domain (Jorissen et al., 2000, Protein Sci. 9:310-324) was superimposed onto the structure determined here for the first modules of the S2 domains of the two sEGFR501 molecules. In the back-to-back dimer the rod-like domains of S2 project towards each other underneath sEGF501, consistent with the ability to form disulfide-linked dimers via a Cys mutation three residues upstream of the transmembrane domain when ligand binds to mutant receptors (Sorokin et al., 1994, J. Biol. Chem. 269:9752-9759). The same superimposition performed on the head-to-head dimer results in the modelled S2 domains projecting away from each other and is inconsistent with the Cys mutant data (Sorokin et al., 1994, J. Biol. Chem. 269:9752-9759).

### Example 8

### Receptor Domain Architecture

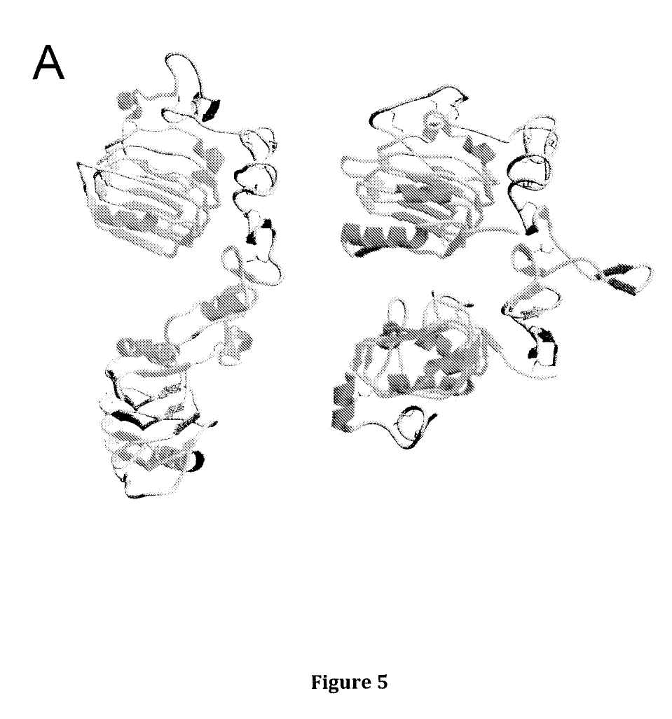

The L1, S1 and L2 domains show both sequence (FIG. 4) and structural (FIG. 5) homology to the first three domains of the type I insulin-like growth factor receptor (Garrett et al., 1998, Nature 394:395-399). More broadly, the L domains resemble other leucine-rich repeat or solenoid proteins (Ward, C. W. and Garrett, T. P. J. 2001, BMC Bioinformatics 2, 4; Kobe B. and Kajava, A. V. 2001, Curr. Opin. Struct. Biol. 11:725-732). Each L domain is composed of six turns of a β-helix or solenoid and is capped at each end by a helix and a disulfide bond. At the C-terminus of the L domains the helix is only vestigial and in each case there is intimate association with the first module of S1 or S2. A conserved Trp from each of these first modules (Trp176 in S1 and Trp492 in S2) is inserted into the body of the L domain between the fourth and fifth turns of the β-helix as seen in IGF-1R (Garrett et al., 1998, Nature 394:395-399), making these modules structurally part of the L domain. In each case the loops in the first cys-rich modules of the S1 and S2 domains of sEGFR501 are shorter than those in IGF-1R and similar in size to the other modules in sEGFR501 (modules 2 and 3 in S1 and 4 and 7 in S2) which contain two disulfide bonds (FIGS. 4A and 4B).

Each of the L domains contains a large β-sheet (second sheet, in FIG. 5), flanked by two shorter ones on either side (blue and yellow). The edge between the first and second β-sheets is characterised by the presence of a stack of conserved Gly residues at positions 39, 63, 85, 122 in L1 and 343, 379, 404 and 435 in L2 (FIG. 4A). The edge at the junction of the second and third β-sheets is formed, in part, by a short Asn ladder as in IGF-1R (Garrett et al., 1998, Nature 394:395-399). A loop from the fourth turn of each solenoid protrudes from the large (second) β-sheet and is common to the EGF and IGF receptor families. Opposite the large β-sheet in both L1 and L2 there is a more irregular face, with the polypeptide strands in the third, fourth and fifth turns in L2 having a similar conformation to those in IGF-1R L1 but different from those in EGFR L1.

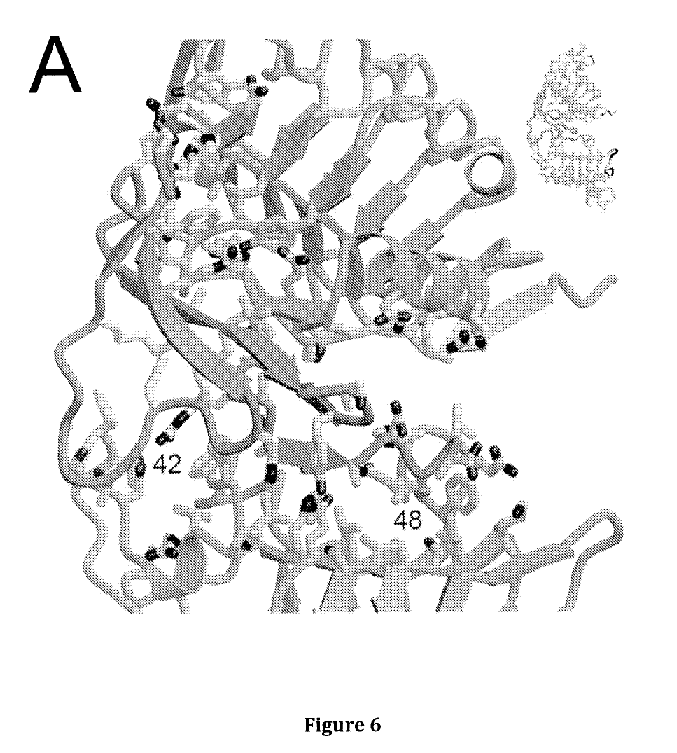

For both L1 and L2 domains of EGFR the long β-strand in the first turn of the solenoid is missing. In L1 this strand is replaced by a long V-shaped excursion (residues 8-18) of the polypeptide chain which sits over the large β-sheet of this domain to form a major part of L1's ligand-binding surface (FIG. 6). In L2 this second strand is replaced by a loop (residues 316-326) which also contacts the ligand (FIG. 6).

The order and association of the eight disulfide-bonded modules in S1 are similar to that of IGF-1R (FIGS. 4A and 4B), with the first module packed against the fourth face of the L1 domain as discussed above and modules 2-8 forming a rod-like domain (FIG. 5) spanning from L1 to L2. Relative to IGF-1R, each of the disulfide bonded modules in sEGFR501 is oriented slightly differently to the previous one (8-36°), with the cumulative effect being that S1 of the EGFR appears as a straight rod, bent at module 6, whereas in IGF-1R the S domain is curved. Even for the two molecules of EGFR in the crystal's asymmetric unit there is a relative difference between modules 6 and 7 of 12°, implying that the modules are not always rigidly associated.

Like IGF-1R, S1 of EGFR makes contact with L1 along one side of the solenoid (sheet 1, burying 1375 Å² of accessible surface area) but in EGFR, S1 also makes appreciable contact with the L2 domain via modules 6 and 7 (burying 860 Å²). This is different to the IGF-1R structure where the L2 domain is rotated away to lie almost perpendicular to the axis of L1 (FIG. 5). Thus the C-terminal region of S1 may act as a hinge in the ligand-free form of the EGFR as modules 7 and 8 appear somewhat mobile, having some of the largest temperature factors in the structure.

The most striking feature of S1 is a large ordered loop from module 5 which projects directly away from the ligand-binding site. The loop consists of residues 242-259 and contains an antiparallel β-ribbon (FIG. 5). This loop is highly conserved within the EGFR family and is different to the insulin receptor family where a loop of similar size points from module 6 into the ligand-binding site (FIG. 5). If EGFR were to have a loop similar to IGF-1R, there would be a substantial steric clash between that loop and L2.

### Example 9

### Structure of TGFα

More than 10 mitogenic peptides form a family of ligands which can bind to members of the EGFR family. However, apart from residues Gly19, Gly40 and the three conserved disulfide bonds which are needed to maintain structure, only Arg42 is conserved throughout the family and pairwise sequence identities between the ligands are often less than 35%. Three-dimensional structures have been determined by NMR for EGF (Montelion et al., 1987, Proc. Natl. Acad. Sci. USA. 84, 5226-5230; Cooke et al., 1987, Nature 327:339-341; Kohda et al., 1992, Biochemistry 31:11928-11939; Barnham, et al., 1998, Protein Sci. 7:1738-1749), TGFα (Tappin et al., 1989, Eur. J. Biochem. 179, 629-637; Harvey et al, 1991, Eur. J. Biochem. 198:555-562; Moy et al., 1993, Biochemistry 32:7334-7353) and heregulin (Nagata et al, 1994, EMBO J. 13:3517-3523; Jacobsen et al, 1996, Biochemistry 35, 3402-3417) and by X-ray crystallography for heparin-binding EGF (HB-EGF) in complex with diphtheria toxin (Louie et al., 1997, Mol. Cell. 1:67-78) and EGF (Lu, et al., 2001, J. Biol. Chem. 276:34913-34917). These structures show that TGFα and its relatives are relatively flexible molecules built on a small structurally conserved core. In particular, the N- and C-terminal residues are often quite disordered. From a comparison of the two molecules of EGF in the asymmetric unit, (Lu, et al., 2001, J. Biol. Chem. 276:34913-34917) found that the common structural core comprised only residues 13-21 and 30-47 (equivalent to 15-22 and 31-48 in TGFα, FIG. 4C) which encompassed half of the large β-ribbon and a small, C-terminal β-ribbon. The structure of TGFα, seen here in the complex, shows substantially more order, with a third, N-terminal β-strand (residues 4-6) aligned with the large β-ribbon (residues 19-33) to form a three-stranded β-sheet and an ordered C-terminus. The structure of TGFα in the 2:2 complex is triangular or crescent shaped. The two TGFα molecules in the dimer superimpose well on each other (rmsd 0.70 for 44 Cα atoms). They are structurally similar to the human EGF molecule A (rmsd 1.33 Å for 41 Cα atoms) in the EGF crystal structure (Lu, et al., 2001, J. Biol. Chem. 276:34913-34917) and even more closely to HB-EGF (0.66 Å for 34 Cα atoms) in its complex with diphtheria toxin (Louie et al., 1997, Mol. Cell. 1:67-78).

### Example 10

### Ligand-Receptor Interactions in the EGF Receptor

In the complex, each sEGFR501 monomer interacts with a single TGFαmolecule and each ligand interacts with the large β-sheets of both the L1 and L2 domains of one receptor molecule (FIGS. 3 and 6). Relative to IGF-1R, the position of L2 corresponds to a rotation by 105′ at the L2/S1 module7 interface or 122-130°, relative to L1 of IGF-1R. More than a third of the ligand's accessible surface area is buried by the L1 and L2 domains of the receptor (about 745 Å² by L1 and about 785 Å² by L2) and over 60% of the ligand's residues make contact with the receptor. The footprint of the ligand on the receptor covers most of the large (second) sheet of each L domain, running from the top left corner to abut the loop in the fourth rung of the solenoid (FIGS. 3 and 6).

In the contact with L1, the inner curved face of the crescent-shaped TGFα sits across the large sheet and extends to the N-terminal helix of L1 (FIG. 6). More than half the buried surface area of L1 comes from a V-shaped loop which runs across the large sheet, replacing the first strand of the corresponding sheet in IGF-1R. In the center of this interface TGFα makes contact with the receptor, primarily via main chain atoms. One strand from the large β-sheet of TGFα (residues 29-35) sits edge on to the receptor and aligns with the latter part of the V-shaped loop (residues 15-17) in L1's first solenoid turn. This enables the receptor to contribute part of the V as a fourth parallel β-strand to the first and larger of the ligand's two β-sheets (FIG. 6). Asn12, which is conserved in all of the EGFR family except ErbB2, makes a side chain to main chain contact with the peptide N atom of Gly40 in TGFα. The Oγ1 atom of Thr15 from L1 also makes a hydrogen bond to Ala41 O of TGFα. This interface is also characterized by a small hydrophobic contact around Leu17 from L1 and hydrophilic and electrostatic interactions involving the ligand's ‘B loop’ residues Arg22, Gln26, Glu27 and Lys29 with the L1 domain residues Tyr45, Tyr101, Arg125, and Glu90 respectively. The location of the N-terminus of TGFα near Tyr101 in the complex is consistent with the chemical cross-linking data of (Woltjer et al., 1992, Proc. Natl. Acad. Sci. USA. 89, 7801-7805). It should be noted that the lack of conservation in ErbB2 of two key residues in this interface (Arg for Thr/Ser at position 15 and Met for Asn at position 12) would prevent any of the EGF family of ligands from binding to L1.

The interface between L2 and TGFα is formed mostly from the side chain atoms of both the ligand and receptor. TGFα sits on the flat face (i.e. the large β-sheet) of L2, surrounded by three loops (residues 316-326, 352-363 and 405-412) which project out from the plane of the sheet (FIG. 6). The contact between the ligand and receptor is an alternating series of stripes of hydrophobic and hydrophilic interaction across the interface. These are as follows: (i) Phe15 of TGFα sits against Phe357 of EGFR; (ii) the strictly conserved Arg42 of TGFα is sandwiched between Phe15 and Phe17 of the ligand facilitating the correct orientation and environment to make a salt bridge with the strictly conserved Asp355 of the receptor; (iii) Phe 17 and the lower part of Glu44 from TGFα interact with Leu325, Leu348 and Val350 from L2; (iv) the next hydrophilic region contains four histidines, His18 and His45 of TGFα and His346 and His409 of L2, as well as Tyr38 and Glu44 from TGFα and Gln384 and Gln408 from L2; and (v) there is a hydrophobic pocket in L2 (Leu382, Gln408, His409, Phe412, Val 417, Ile438), centred over Ala415, which holds the highly conserved Leu48 of TGFα (Leu47 in EGF), the ligand residue with the largest buried surface The C-terminus of TGFα is sandwiched between domains L1 and L2, with the side chain of Leu49 contacting both L domains. Leu49 may well define the final positioning of the L domains in the complex. Lys465 from L2 is near the C-terminus of TGFα and may stabilise the terminal carboxyl group. Lys465 has been chemically cross-linked to residue 45 in a mutant form of mouse EGF (Summerfield et al., 1996, J. Biol. Chem. 271:19656-19659). Some carbohydrate nearby could possibly also affect ligand binding.

There appears to be a number of key contacts, with the ionic interaction between TGFα Arg42 and EGFR Asp355 and the hydrophobic interaction between TGFα Leu48 and the hydrophobic pocket centred over EGFR Ala415 being particularly important. These features are conserved in all ErbB family members.

Although the interactions of EGFR with TGFα are ostensibly the same for both molecules in the crystal's asymmetric unit, it should be noted that when the ligands are superimposed, the L1 domains differ by a rotation of 3.5° about Leu14 Cy and for the L2 domain approximately 8° about Ala415 Cβ in EGFR and the side chain of Leu48 in TGFα. These observations suggest that while there may be a bit more flexibility in the TGFα:L2 interface, Leu48 is the major determinant of ligand binding to L2. The cluster of His residues in the middle of the L2 interface may play a part in release of the ligand at low pH following endocytosis.

### Example 11

### Receptor-Receptor Interactions

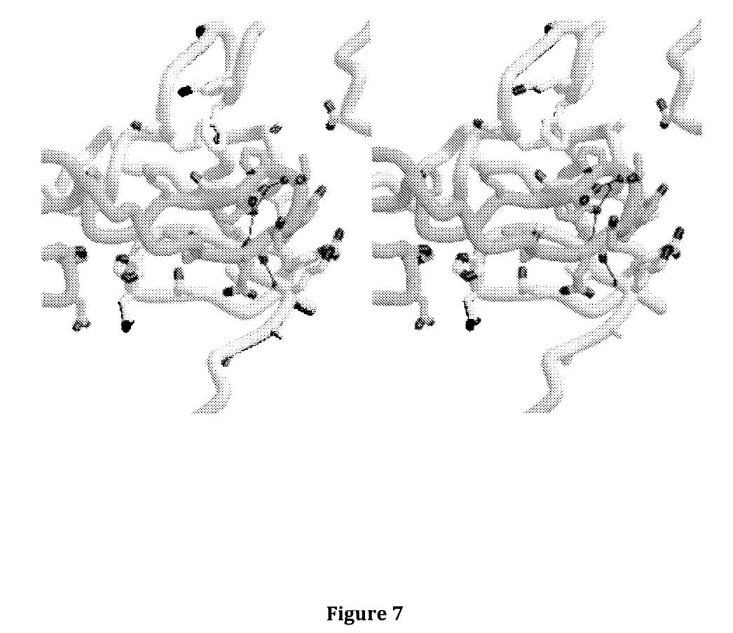

Unlike other growth factor receptor complexes, the ligand is not found at the dimer interface in the 2:2 complex of TGFα:sEGFR501. Thus ligand induced dimerization of sEGFR501 implies that binding of ligand induces a conformational change in the receptor that promotes receptor-receptor interactions. The most notable feature of the back-to-back dimer is a long loop (residues 242-259) which is specific to the EGFR family and is not found in the CR of IGF-1R (FIGS. 4B and 5) or other members of the insulin receptor family. From each receptor the loop projects out from the fifth module of S1, across the other S1 domain to a space between L1, L2 and S1 domains of the neighbouring receptor (FIG. 3). Contact is made by residues 244-253 of the S1 loop in, say, molecule A with residues 229-239, 262-278, and 282-288 on the concave face of the S1 domain of molecule B (FIG. 3). The buried surface areas are 480 Å² and 330 Å², respectively. At specific positions in the S1 loop there is remarkable sequence conservation across all ErbB family members. Tyr246 is strictly conserved and is completely buried in the interface. The Oη atom of TyrA246 (receptor molecule A) makes hydrogen bonds with the GlyB264 N and CysB283 O atoms (receptor molecule B) and the phenyl ring sits against the Cp atoms of SerB262 and SerB282 and the face of the following peptides (FIG. 7). Residue 251 is strictly conserved as Tyr or Phe and in this interface makes a hydrophobic contact via the benzene ring with the PheB263, GlyB264, TyrB275 and ArgB285. The Oη of TyrA251 is exposed to solvent. Additional hydrophobic contacts are made by ProA248 to PheB230 and AlaB265; and by MetA253 to ThrB278. There is also a hydrogen bond from TyrA251 O to ArgB285 N (FIG. 7).

Other conserved residues of the S1 loop, such as Asn247 and Asn256, do not make contact with the other half of the dimer, but hydrogen bond back onto the main chain and appear to be important for maintaining the loop in the appropriate conformation. There are four positions in the loop (residues 243, 248, 255 and 257) where proline is found in at least one member of the human EGFR family with ErbB3 having as many as three prolines. These prolines would further stabilise the conformation of the loop.

The loop not only touches the S1 domain of its partner, but also reaches across to contact the L1 and L2 domains of the other receptor molecule (burying a surface area of 40 Å² on L1 and 5 Å² on L2). AsnB86 touches ThrA249 and, with a slight rearrangement, could form a hydrogen bond between the side chains. Neither residue is conserved in other ErbB receptors although polar residues predominate at these positions. ThrA250, which is conserved in other ErbB receptors, sits near IleB318 but the reason for the conservation is not apparent. Although these interactions are quite weak, it is possible that the binding of the loop from one receptor may be affected by binding of ligand to the other, as ligand binding may alter the relative positions of the L domains.

Two other regions also participate in the back-to-back dimer contact. One is near the two long loops, where Asp279 and His280 of receptor A make contact across the dimer axis with the corresponding residues from receptor B (FIG. 3). A second region of contact is near the N-terminal end of the S1 domain in cys-rich module 2, where residues 193-195 and 204-205 from molecule A contact 193-194 and 204-205 from molecule B, burying about 225 Å².

### Example 12

### Functional Characterisation of Mutant EGFRs Expressed in BaF/3 Cells

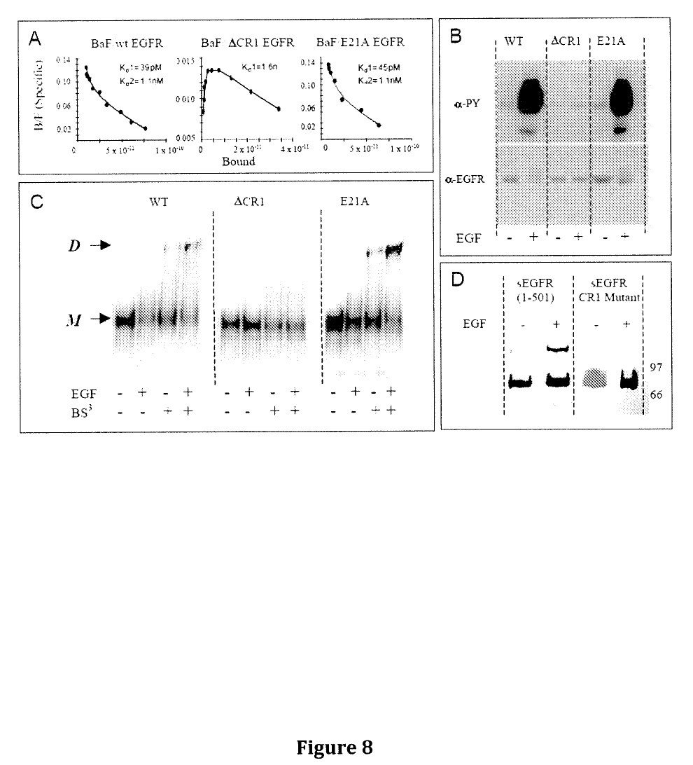

In order to establish the biological relevance of the two dimers identified in crystals of the TGFα:sEGFR501 complex, mutant receptors designed to probe the two dimer interfaces were analyzed. Single amino acid substitutions Glu21Ala, Arg470Leu, Asn473Asp, Ser474Glu and Ala477Asp were prepared to test the head-to-head dimer. When transiently expressed in 293 cells, which express low endogenous levels of EGFR (<1×10⁴ receptors/cell), or when stably expressed (Glu21Ala) in the hemopoietic cell line BaF/3 which do not express EGFR family members (Walker et al., 1998, Growth Factors 16:53-67), these mutants showed normal EGF binding, kinase activation, dimerization (FIG. 8) and internalization (data not shown). In contrast mutants of the back-to-back dimer, an S1 loop deletion (residues Δ242-259) from the full length receptor and sEGFR501 with multiple substitutions in the S1 loop (Tyr246Asp, Asn247Ala, Thr249Asp, Tyr251Glu, Gln252Ala and Met253Asp) were defective. The ΔS1-loop clones fail to show ligand-induced dimerization and ligand-induced kinase activation and exhibit only low affinity binding (FIGS. 8A, B, C). The sEGFR501 mutants fail to show ligand-induced dimerization (FIG. 8D) and exhibit 15 fold lower affinity binding on BIAcore (500 nM vs 30 nM for sEGFR501).

### CONCLUSION

Ligand-induced dimerisation (or oligomerisation) of receptors is a common means of signal transduction and in all cases seen so far the ligand participates directly in the dimerisation of receptors. For VEGF/Flt-1 (Wiesmann et al., 1997, Cell 91:695-704), nerve growth factor (NGF)/TrkA receptor (Weismann et al., 1999, Nature 401:184-188.), bone morphogenic protein (BMP)/BMP receptor (Kirsch et al., 2000, Nat. Struct. Biol. 7:492-496), interferon γ(IFNγ)/IFNγ receptor (Thiel et al., 2000, Structure Fold Des. 8:927-936) and tumour necrosis factor (TNF)/TNF receptor (Banner et al., 1993, Cell 73:431-445), the ligand is a dimer or trimer before forming the 2:2 complex or 3:3 complex, and in the structures determined, the receptors do not contact each other. In the 2:2 complex of the fibroblast growth factor (FGF)/FGF receptor the ligands do not contact each other but are dimerised by heparin (Plotnikov et al., 2000, Cell 101:413-424; Schlessinger et al., 2000, Molecular Cell 6:743-750; Sorokin et al., 1994 J. Biol. Chem. 269:9752-9759; Pelligrini et al., 2000, Nature 407:1029-1034). The FGF receptors do contact each other and the two FGF ligands lie at the dimer interface with a heparin molecule sitting between two FGFs. In the 2:2 complex of granulocyte colony stimulating factor (GCSF)/GCSF receptor (Aritomi et al., 1999, Nature 401:713-715) each ligand binds both receptors but there are no contacts between the two ligands or the two receptor fragments. Finally, in the growth hormone, erythropoietin and prolactin/receptor complexes, there is only one ligand molecule in the 1:2 complex and the two receptor molecules make contact with ligand and with each other (de Vos et al., 1992, Science 255:306-312).

The TGFα:EGFR complex represents a new and surprising way in which receptors and protein ligands interact. EGFR ligands bind at a site remote from the dimer interface and must modify the receptor to promote dimerisation. A precedent for this has been seen for much smaller ligands. For example, in the rat metabotrophic glutamate receptor, a disulfide-linked homodimer, binds glutamate between two domains of the receptor monomer, causing them to go from an ‘open’ to a ‘closed’ form (Kunishima et al., 2000, Nature 407:971-977). Such a mechanism could also occur in the EGFR family where the ligand binds both L1 and L2, fixing the relative orientations of the two domains. Compared to IGF-1R there is a substantial rearrangement of L domains in EGFR (FIG. 5) although a conformational change of such a magnitude would not be necessary. A smaller change in L domain positions upon ligand binding, possibly with hinge motions seen at the S1 module 5/6, 6/7 and 7/L2 interfaces (relative to IGF-1R), could enable EGFR extracellular domains to form dimers.

The disclosure of all publications referred to in this application are include herein by reference.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

TABLE 1
Summary of crystallographic data

| | | | | Completeness (%) | | | | |
|---|---|---|---|---|---|---|---|---|
| Data set | Resolution (Å) | Mean I/s | R_{merge}* | (Multiplicity) | No of sites | R_{Cullis}^{†} | Phasing Power^{‡} | f.o.m.^{§} |
| Native | 2.9 | 11.1 | 0.129 | 96.9 (2.78) | | | | 0.31/0.84 |
| Pt(NO₃)₂ | 2.8 | 11.9 | 0.095 | 97.8 (3.85) | 4 | 0.71 | 0.71 | |
| PIP | 2.5 | 10.8 | 0.075 | 90.2 (3.17) | 2 | 0.91 | 0.91 | |
| K₂Au(CN)₂ | 3.0 | 9.1 | 0.091 | 97.8 (3.43) | 4 | 0.21 | 2.21 | |

| Refinement | | | | | | |
|---|---|---|---|---|---|---|
| Resolution (Å) | No. of reflections (free) | No. of atoms | R_{cryst}^{#} | R_{free}^{#} | Bonds^{¶}(Å) | Angles^{¶}(°) |
| 20-2.5 | 48006 (2379) | 8687 | 0.237 | 0.289 | 0.007 | 1.50 |

PIP,di-μ-iodobis(ethylenediamine)diplatinum nitrate (Unit cell a = 52.02 Å, b = 198.17 Å, c = 78.43 Å, β = 102.95°)
*R_{merge} = ΣₕΣⱼ|Iₕⱼ-Iₕ|/ΣₕΣⱼIₕ, where Iₕⱼ is an intensity measurement j and Iₕ is the mean for a reflection h.
^{†}R_{Cullis} = Σₕ||F_{PH}-F_{P}|-|F_{Hcalc}||/Σₕ||F_{PH}|-|F_{P}||, where F_{PH}, Fₚ and F_{Hclac} are, respectively, derivative, native and heavy atom structure factors for centric reflection h.
^{‡}Phasing power = Σₕ|F_{Hcalc}|/Σₕε, where F_{Hcalc} is defined above and ε is the lack of closure.
^{§}f.o.m.(figure of merit) = <cos(Δαₕ)>, where Δαₕ is the error in the phase angle for reflection h. Values are given before and after density modification.
^{#}R_{cryst} and R_{free} are defined in.
^{¶}R.m.s. deviation for bond distances and angles.

1. An antibody which binds to an EGF receptor family member, the antibody being directed against:
(a) in the case of EGFR (i) EGFR residues 100-108, 315-327 or 353-362; or
(ii) EGFR residues 190-207, 240-305 or parts thereof;
(b) in the case of ErbB-2 (i) ErbB-2 residues 98-116, 323-335 or 361-374; or
(ii) ErbB-2 residues 198-214, 247-313 or parts thereof;
(c) in the case of ErbB-3 (i) ErbB-3 residues 103-112, 314-324 or 350-363; or
(ii) ErbB-3 residues 190-207, 240-304 or parts thereof; and
(d) in the case of ErbB-4 (i) ErbB-4 residues 102-111, 316-328, 354-367; or
(ii) ErbB-4 residues 192-209, 242-306 or parts thereof.

2. The antibody of claim 1, the antibody being directed against EGFR residues 240-305 or part thereof.

3. The antibody of claim 1, the antibody being directed against ErbB-2 residues 247-313 or part thereof.

4. The antibody of claim 1, the antibody being directed against ErbB-3 residues 240-304 or part thereof.

5. The antibody of claim 1, the antibody being directed against ErbB-4 residues 242-306 or part thereof.

6. The antibody of claim 1, wherein the antibody interacts with a member of the EGF receptor family in a manner such as to interfere with the formation of active dimers by inhibiting interaction of;
(i) one or more residues selected from the group consisting of residues 38, 86, 194, 195, 204, 205, 230, 239, 242-246, 248-253, 262-265, 275, 278-280, 282-288 and 318 of EGFR or the corresponding region of a member of the EGF receptor family other than EGFR;
with
(ii) one or more residues selected from the group consisting of residues 86, 193, 194, 204, 205, 229, 230, 239, 242, 244-246, 248-253, 262-265, 275, 278-280 and 282-287 of EGFR or the corresponding region of a member of the EGF receptor family other than EGFR.

7. The antibody of claim 6, wherein the antibody interferes with the formation of homodimers.

8. The antibody of claim 6, wherein the antibody interferes with the formation of heterodimers.

9. The antibody according to claim 6, wherein the antibody interacts with a member of the EGF receptor family in a manner such as to interfere with the formation of active dimers by inhibiting interaction of;
(i) one or more residues selected from the group consisting of residues 36, 84, 201-203, 211, 212, 236, 237, 246, 249-253, 255-260, 269-272, 282, 285-287, 289-295 and 326 of ErbB-2;
with
(ii) one or more residues selected from the group consisting of residues 38, 86, 193-195, 204, 205, 229, 230, 239, 242-246, 248-253, 262-265, 275, 278-280, 282-288 and 318 of EGFR or the corresponding region of a member of the EGF receptor family other than EGFR.

10. The antibody according to claim 6, wherein the antibody interacts with a member of the EGF receptor family in a manner such as to interfere with the formation of active dimers by inhibiting interaction of;
(i) one or more residues selected from the group consisting of residues 41, 89, 193-195, 204, 205, 229, 230, 239, 242-246, 248-253, 262-265, 275, 278-279, 281-287 and 317 of ErbB-3;
with
(ii) one or more residues selected from the group consisting of residues 38, 86, 193-195, 204, 205, 229, 230, 239, 242-246, 248-253, 262-265, 275, 278-280, 282-288 and 318 of EGFR or the corresponding region of a member of the EGF receptor family other than EGFR.

11. The antibody according to claim 6, wherein the antibody interacts with a member of the EGF receptor family in a manner such as to interfere with the formation of active dimers by inhibiting interaction of;
(i) one or more residues selected from the group consisting of residues 40, 88, 195-197, 206, 207, 231, 232, 241, 244-248, 250-255, 264-267, 277, 280-281, 283-289 and 319 of ErbB-4;
with
(ii) one or more residues selected from the group consisting of residues 38, 86, 193-195, 204, 205, 229, 230, 239, 242-246, 248-253, 262-265, 275, 278-280, 282-288 and 318 of EGFR or the corresponding region of a member of the EGF receptor family other than EGFR.

12. The antibody of claim 1, wherein the antibody has a Kd for the receptor site of less than 10⁻⁶M.

13. The antibody of claim 1, wherein the antibody has a Kd for the receptor site of less than 10⁻⁸M.

14. A pharmaceutical composition for preventing or treating a disease associated with signalling by a molecule of the EGF receptor family which comprises an antibody of claim 1 and a pharmaceutically acceptable carrier or diluent.


SEQUENCE LISTING (27 sequences)

SEQ ID NO: 1 - protein, 501 aa, Homo sapiens
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVS

SEQ ID NO: 2 - protein, 509 aa, Homo sapiens
STQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVN

SEQ ID NO: 3 - protein, 500 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLS

SEQ ID NO: 4 - protein, 502 aa, Homo sapiens
QPSDSQSVCAGTENKLSSLSDLEQQYRALRKYYENCEVVMGNLEITSIEHNRDLSFLRSVREVTGYVLVALNQFRYLPLENLRIIRGTKLYEDRYALAIFLNYRKDGNFGLQELGLKNLTEILNGGVYVDQNKFLCYADTIHWQDIVRNPWPSNLTLVSTNGSSGCGRCHKSCTGRCWGPTENHCQTLTRTVCAEQCDGRCYGPYVSDCCHRECAGGCSGPKDTDCFACMNFNDSGACVTQCPQTFVYNPTTFQLEHNFNAKYTYGAFCVKKCPHNFVVDSSSCVRACPSSKMEVEENGIKMCKPCTDICPKACDGIGTGSLMSAQTVDSSNIDKFINCTKINGNLIFLVTGIHGDPYNAIEAIDPEKLNVFRTVREITGFLNIQSWPPNMTDFSVFSNLVTIGGRVLYSGLSLLILKQQGITSLQFQSLKEISAGNIYITDNSNLCYYHTINWTTLFSTINQRIVIRDNRKAENCTAEGMVCNHLCSSDGCWGPGPDQCLS

SEQ ID NO: 5 - protein, 53 aa, Homo sapiens
NSDSECPLSHDGYCLHDGVCMYIEALDKYACNCVVGYIGERCQYRDLKWWELR

SEQ ID NO: 6 - protein, 52 aa, Homo sapiens
VVSHFNDCPDSHTQFCFHGTCRFLVQEDKPACVCHSGYVGARCEHADLLAVV

SEQ ID NO: 7 - protein, 44 aa, Homo sapiens
KKKNPCNAEFQNFCIHGECKYIEHLEAVTCKCQQEYFGERCGEK

SEQ ID NO: 8 - protein, 50 aa, Homo sapiens
KKRDPCLRKYKDFCIHGECKYVKELRAPSCICHPGYHGERCHGLSLPVEN

SEQ ID NO: 9 - protein, 50 aa, Homo sapiens
GHFSRCPKQYKHYCIKGRCRFVVAEQTPSCVCDEGYIGARCERVDLFYLR

SEQ ID NO: 10 - protein, 50 aa, Homo sapiens
VSITKCSSDMNGYCLHGQCIYLVDMSQNYCRCEVGYTGVRCEHFFLTVHQ

SEQ ID NO: 11 - protein, 50 aa, Homo sapiens
KFSHPCLEDHNSYCINGACAFHHELKQAICRCFTGYTGQRCEHLTLTSYA

SEQ ID NO: 12 - protein, 53 aa, Homo sapiens
SHLVKCAEKEKTFCVNGGECFMVKDLSNPSRYLCKCQPGFTGARCTENVPMKV

SEQ ID NO: 13 - protein, 53 aa, Homo sapiens
SHLVKCAEKEKTFCVNGGECFMVKDLSNPSRYLCKCPNEFTGDRCQNYVMASF

SEQ ID NO: 14 - protein, 50 aa, Homo sapiens
GHARKCNETAKSYCVNGGVCYYIEGINQLSCKCPNGFFGQRCLEKLPLRL

SEQ ID NO: 15 - protein, 50 aa, Homo sapiens
GHARKCNETAKSYCVNGGVCYYIEGINQLSCKCPVGYTGDRCQQFAMVNF

SEQ ID NO: 16 - protein, 52 aa, Homo sapiens
EHFKPCRDKDLAYCLNDGECFVIETLTGSHKHCRCKEGYQGVRCDQFLPKTD

SEQ ID NO: 17 - protein, 50 aa, Homo sapiens
DHEQPCGPRHRSFCLNGGICYVIPTIPSPFCRCIENYTGARCEEVFLPSS

SEQ ID NO: 18 - protein, 186 aa, Homo sapiens
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKL

SEQ ID NO: 19 - protein, 183 aa, Homo sapiens
EICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQK

SEQ ID NO: 20 - protein, 161 aa, Homo sapiens
KVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCES

SEQ ID NO: 21 - protein, 191 aa, Homo sapiens
KVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVS

SEQ ID NO: 22 - protein, 124 aa, Homo sapiens
TKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCR

SEQ ID NO: 23 - protein, 116 aa, Homo sapiens
MCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCP

SEQ ID NO: 24 - protein, 121 aa, Homo sapiens
SCRNVSRGRECVDKCKLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPS

SEQ ID NO: 25 - protein, 50 aa, Homo sapiens
VVSHFNDCPDSHTQFCFHGTCRFLVQEDKPACVCHSGYVGARCEHADLLA

SEQ ID NO: 26 - protein, 53 aa, Homo sapiens; identical to SEQ ID NO: 5

SEQ ID NO: 27 - protein, 48 aa, Homo sapiens
LGKKRDPCLRKYKDFCIHGECKYVKELRAPSCICHPGYHGERCHGLSL